(19) United States
(12) Patent Application Publication
(10) Pub. No.: US 2009/0181397 A1
(43) Pub. Date: Jul. 16, 2009

(54) PREDICTIVE AND DIAGNOSTIC METHODS FOR CANCER

(75) Inventors: Jin-Tang Dong, Lilburn, GA (US); Xue-Yuan Dong, Decatur, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: Emory University, Atlanta, GA (US)

(21) Appl. No.: 12/354,009

(22) Filed: Jan. 15, 2009

Related U.S. Application Data
(60) Provisional application No. 61/021,098, filed on Jan. 15, 2008.

(51) Int. Cl. C12Q 1/68; C12N 5/00

(52) U.S. Cl. 435/6; 435/377

(57) ABSTRACT

The present disclosure encompasses methods of diagnosing the presence of a cancer, and particularly a cancer of prostate or breast tissue, in a human subject, predicting the outcome or severity of the disease and methods of reversing the prostate cell transformation based on the presence or absence in the human subject of a dinucleotide (TT) deletion in the gene encoding the U50 snoRNA. Provided, therefore, are methods of identifying a genetic marker of a human subject indicating a cancerous tissue in the human subject, embodiments of the methods comprising: determining from an isolated nucleic acid sample the genotype of the human subject with respect to a locus encoding a snoRNA U50, where a mutation within the nucleotide sequence encoding a snoRNA U50, when compared with a wild-type nucleotide sequence encoding a snoRNA U50, identifies in the human subject a genetic marker associated with a cancer in the human subject. The cancer may be, but is not necessarily limited to, a prostate cancer or a breast cancer.

### CROSS-REFERENCE TO RELATED APPLICATIONS

This application claims priority to U.S. Provisional Patent Application Ser. No. 61/021,098, entitled “Predictive and Diagnostic Methods for Prostate Cancer” filed on Jan. 15, 2007, the entirety of which is hereby incorporated by reference.

### STATEMENT REGARDING FEDERALLY SPONSORED RESEARCH OR DEVELOPMENT

This invention was made with government support under NIH Grant No. RO1 CA085560 awarded by the U.S. National Institutes of Health of the United States government. The government has certain rights in the invention.

### TECHNICAL FIELD

The present disclosure is generally related to genetic lesions associating with cancerous cells, and in particular to prostate and breast cancer cells. The present disclosure further relates to methods of detecting a cancer or predicting the prognostic outcome of a cancer as determined by the genotype of the human subject with respect to snoRNA U50.

### BACKGROUND

Prostate cancer is the most common non-skin cancer in the developed regions of the world. The majority of prostate cancers, however, do not present clinical symptoms during a man's natural life and are considered indolent or clinically insignificant (Scardino et al., (1992) Hum. Pathol. 23: 211-222; Sakr et al., (1994) In Vivo 8: 439-443). With widespread prostate-specific antigen (PSA) testing, many indolent prostate cancers are unnecessarily detected (Postma & Schroder (2005) Eur. J. Cancer 41: 825-833), and as many as seven of eight human subjects with screen-detected prostate cancer could be unnecessarily treated (Mcgregor et al., (1998) Can. Med. Assoc. J. 159: 1368-1372). An important question is which men with prostate cancer should be treated and who are better served by merely watchful waiting.

Prostate cancer is considered a multistep disease resulting from the accumulation of genetic alterations including activation of oncogenes and inactivation of tumor suppressor genes. Identification and characterization of genetic alterations underlying prostate cancer could help not only in detecting clinically significant prostate cancers but also in understanding prostate cancer biology. Chromosomal deletion is a hallmark of tumor-suppressor genes because it can reveal recessive mutations, cause haploid sufficiency or truncate/abolish a gene through loss of heterozygosity, hemizygous deletion or homozygous deletion, respectively. Many chromosomal regions are frequently deleted in human cancer, as demonstrated by various genetic approaches, but the affected genes for most of them are still unknown (Knuutila et al., (1999) Am. J. Pathol. 155: 683-694; Dong J. T. (2001) Cancer Metastasis Rev. 20: 173-193). Deletion of chromosome 6 involving q14-q22 is one of the most common deletions in different types of human cancers including prostate cancer (Knuutila et al., (1999) Am. J. Pathol. 155: 683-694; Dong J. T. (2001) Cancer Metastasis Rev. 20: 173-193). Functionally, chromosome 6 transferred into cancer cells induces senescence, reduces cell growth, inhibits tumorigenicity and decreases metastatic potential (Trent et al., (1990) Science 247: 568-571; Welch et al., (1994) Oncogene 9: 255-262; Theile et al., (1996) Oncogene 13: 677-685; Morelli et al., (1997) Cytogenet. Cell genet 79: 97-100; Miele et al., (2000) Int. J. Cancer 86: 524-528)). While these studies indicated the possible existence of one or more tumor-suppressor genes in 6q, such critical genes had not been identified.

Breast cancer is the most common major cancer among women in the United States and breast cancer is the leading cause of cancer deaths in women, second only to lung cancer. An estimated 200,000 cases of breast cancer are diagnosed each year and more 43,300 lives are claimed in consequence. Significantly, in their lifetime, women of all ages have a one in eight chance of developing breast cancer. In consequence, early detection of breast cancer remains paramount to the survivability of victims of breast cancer.

In breast cancers, prognosis is determined primarily by the presence or absence of metastases in draining axillary lymph nodes. However, in approximately one third of women with breast cancer who have negative lymph nodes, the disease recurs and about one third of human subjects with positive lymph nodes are free of disease ten years after local or regional therapy. Furthermore, an increasing proportion of breast cancers are being diagnosed at an early stage because of increased awareness and wider use of screening modalities. Universal application of systematic therapy to these human subjects can lead to over-treatment. According to the St Gallen and NIH consensus, 70-80% of the Stage I and II human subjects would not have developed distant metastases without adjuvant treatment and may potentially suffer from the side effects. These data highlight the need for more sensitive and specific prognostic assays that could significantly reduce the number of human subjects that receive unnecessary treatment.

Tumor size and lymphatic or vascular invasion have been found to be of significant prognostic value in several studies. Quantitative pathological features, i.e. nuclear morphology, DNA content and proliferative activity may further demarcate tumors that have a high chance of micrometastases. Known molecular genetic changes that affect human subject outcome include Her2/NEU over-expression, DNA amplifications, p53 mutations, ER/PR status, uPA and PAI expression. Because the metastatic cascade is a complex process that includes multiple steps, single factors that contribute to tumor process have limitations for prognostic assessment.

### SUMMARY

SnoRNA U50 has a homozygous 2 bp deletion in approximately 10% of sporadic prostate cancers. The homozygous genotype of the deletion is also significantly associated with clinically significant prostate cancer in a prospectively analyzed cohort of prostate cancer cases and controls. The findings support that snoRNA U50 is a 6q14-15 tumor suppressor gene in human prostate cancer, its homozygous deletion is involved in approximately 10% of sporadic prostate cancers and that germline homozygosity of the deletion could predict clinically significant prostate cancer.

The present disclosure encompasses methods of diagnosing the presence of a cancer, and particularly a cancer of prostate or breast in a human subject, predicting the occurrence of a prostate or breast cancer in an individual or the general population, predicting the outcome or severity of the disease and methods of reversing the prostate cell transformation based on the presence or absence in the human subject of a dinucleotide (TT) deletion in the gene encoding the U50 snoRNA.

One aspect of the present disclosure, therefore, provides methods of identifying a genetic marker of a human subject indicating a cancerous tissue in the human subject, embodiments of the methods comprising: obtaining an isolated nucleic acid sample from a human subject; and determining from the isolated nucleic acid sample the genotype of the human subject with respect to a locus encoding a snoRNA U50, whereby a mutation within the nucleotide sequence encoding a snoRNA U50, when compared with a wild-type nucleotide sequence encoding a snoRNA U50, identifies in the human subject a genetic marker associated with a cancer in the human subject. The cancer may be, but is not necessarily limited to, a prostate cancer or a breast cancer.

In the various embodiments of the disclosure, the step of determining from the isolated nucleic acid the genotype of the biological sample with respect to a U50 locus encoding a snoRNA U50 may comprise: isolating by PCR amplification a nucleic acid and determining whether the nucleic acid molecule has a dinucleotide deletion when compared to a wild-type control nucleotide sequence.

The methods may further comprise correlating the presence of the genetic marker in the gene locus encoding the snoRNA U50 with the prognostic outcome for a prostate cancer in the human subject. In some embodiments of the disclosure, the methods may further comprise correlating the presence of the genetic marker in the gene locus encoding the snoRNA U50 with the presence or absence of a breast cancer in the human subject.

Another aspect of the disclosure provides a method of modifying the proliferative status of a cell by introducing into the cell a nucleic acid molecule comprising a sequence comprising the sequence of nucleotides from nucleotide about position 47 to about position 60 of the nucleotide sequence according to SEQ ID NO: 1. In embodiments of this aspect of the disclosure, the nucleic acid molecule may comprise the nucleotide sequence according to SEQ ID NO: 1. The introduction into the cell of the nucleic acid molecule may reduce the proliferation of the cell such as a prostate cancer cell or a breast cancer cell.

Another aspect of the disclosure provides embodiments of a kit for determining whether a biological sample from a human subject has dinucleotide deletion within a nucleic acid region encoding the snoRNA U50, wherein the kit may comprise at least one oligonucleotide comprising a nucleotide sequence selected from the group consisting of: the nucleotide sequences according to SEQ ID NOs: 3, 4, 5, 6, 7, 18, and 19, and instructions for determining whether an isolated nucleic acid sample from a human subject has a cancer-associated mutation within a nucleotide region encoding snoRNA U50.

### BRIEF DESCRIPTION OF THE FIGURES

Further aspects of the present disclosure will be more readily appreciated upon review of the detailed description of its various embodiments, described below, when taken in conjunction with the accompanying drawings.

FIGS. 1A-1C are digital images of gel electrophoretic patterns of the products derived from duplex PCR illustrating the mapping of the deletion region in 6q14.3-q15 in prostate cancer. FIG. 1A: homozygous deletion detected by duplex PCR in xenograft LuCaP 73; FIG. 1B: hemizygous deletion detected by duplex PCR in xenograft LuCaP 105; FIG. 1C: hemizygous deletion detected by duplex PCR in xenograft LAPC3. Sample names are at the top, markers at the left, and the sizes (bp) of the PCR products are indicated at the right.

FIG. 2 illustrates the deletion status for each marker and the definition of the minimal region of deletion at 6q14-15 in prostate cancer. The normal sample was from a normal human placenta. Marker names are at the top, sample names at the left and the minimal region of deletion is marked by a horizontal line at the bottom. The sequence map is indicated for each marker. ‘O’, homozygous deletion; ‘−’, hemizygous deletion; ‘+’, no deletion. The location of U50 is indicated by an arrow.

FIG. 3A illustrates the nucleotide sequence (SEQ ID NO: 1) of the wild-type snoRNA U50. The nucleotides harboring the dinucleotide deletion (TT) are marked by asterisks.

FIG. 3B illustrates the nucleotide sequence (SEQ ID NO: 2) of the ΔTT snoRNA U50 variant.

FIG. 4 is a digital image of a northern blot analysis illustrating the reduced expression of snoRNA U50 in prostate cancer samples. Sample names are shown at the top. The 28S RNA indicates relative amounts of total RNA loaded into each lane.

FIG. 5 shows a graph illustrating the reduced expression of snoRNA U50 in prostate cancer cell lines and xenografts as shown by real-time PCR analysis. Sample names are at the bottom, and U50 expression in each sample was normalized to that in normal prostates.

FIG. 6 shows a graph illustrating the reduced expression of snoRNA U50 in 15 localized tumors of prostate cancer, as determined by real-time PCR analysis. Sample case numbers are at the bottom, and U50 expression in each sample was matched to normal tissue from human subject 1.

FIG. 7 is a digital image of a northern blot analysis illustrating the verification of U50 expression upon the transfection of U50 expression plasmid into 22Rv1 and LNCaP cells.

FIG. 8 is a digital image illustrating a six-well plate showing U50-expression-reduced colony number in 22Rv1 cells at 12 days after transfection.

FIG. 9 is a graph illustrating cell numbers estimated by the SRB staining and the measurement of optical densities (y axis) after U50 transfection into LNCaP and 22Rv1 cells. The readings were from day 12 post-transfection. *P<0.005.

FIG. 10 is a graph illustrating the expression of U50′ did not alter colony forming efficiency, whereas a mixture of U50 and U50′ still did. *P=0.547; **P=0.037.

FIG. 11 is a graph illustrating that the TT deletion abolished the function of U50 in suppressing colony formation in LNCaP cells. *P<0.005 when compared with vector control; **P=0.44 when compared with vector control, but P<0.005 when compared with wild-type U50 control.

FIG. 12 is a graph illustrating that the TT deletion abolished the function of U50 in suppressing the proliferation of LNCaP cells. *P=0.007 when compared with vector control; **P=0.457 when compared with vector control, but *P=0.007 when compared with wild-type U50.

FIG. 13 is a digital image of a sequencing gel electrophoresis pattern of PCR products illustrating the detection of a U50 mutation in prostate cancer at the genomic DNA level. Xenografts LAPC3 and LuCaP 96 show a homozygous genotype for the TT deletion in U50 (U50ΔTT), whereas cell line NCI-H660 and xenograft LuCaP 86.2 are heterozygous for the deletion. Sample names or case numbers are at the top. T: tumor cells; N: matched non-cancer cells.

FIG. 14 shows graphical images illustrating DNA sequencing results showing wild-type, homozygous, and heterozygous mutants of U50 in a normal sample, xenograft LAPC3 and xenograft LuCaP 86.2, respectively. Arrows point to the affected nucleotides. Sample names or case numbers are at the top, and all mutations were detected by sequencing gel electrophoresis of PCR products. T: tumor cells; N: matched non-cancer cells.

FIG. 15 is a digital image of a sequencing gel electrophoresis of PCR products illustrating somatic mutations of U50 in three primary prostate cancers. Lower panels show representative results from a STR (short tandem repeat) marker verifying the same origin of normal and cancer cells for each of the cases, as detected by the AmpFLSTR Identifiler PCR Amplification Kit. Sample names or case numbers are at the top, and all mutations were detected by sequencing gel electrophoresis of PCR products. T: tumor cells; N: matched non-cancer cells.

FIG. 16 is a digital image of a sequencing gel electrophoresis pattern of PCR products illustrating the homozygous genotype of the TT deletion detected in both cancer and normal cells from two prostate cancer human subjects. Sample names or case numbers are at the top, and all mutations were detected by sequencing gel electrophoresis of PCR products. T: tumor cells; N: matched non-cancer cells.

FIG. 17 is a digital image of a sequencing gel electrophoresis pattern of PCR products illustrating the tumor-specific loss of the wild-type allele in four cases that had a heterozygous genotype for the TT deletion. Lower panels show representative results from an STR marker verifying the same origin of normal and cancer cells for each case. Sample names or case numbers are at the top, and all mutations were detected by sequencing gel electrophoresis of PCR products. T: tumor cells; N: matched non-cancer cells.

FIG. 18A is a graph illustrating the extent of chromosomal deletions of the U50 locus in cell lines derived from breast cancer as detected by real time PCR.

FIG. 18B is a digital image of a gel electrophoretic analysis of the extent of chromosomal deletions of the U50 locus in cell lines derived from breast cancer as detected by duplex PCR.

FIG. 19 is a graph comparing the expression of U50 snoRNA in various breast cancer cell lines as detected by real time PCR. Samples with the homozygous U50 TT-deletion are marked by an asterisk (*).

FIG. 20 shows digital images of denaturing gel electrophoretic analyses of U50 homozygous mutations in the breast cancer cell lines Hs 578T, MDA-MB-231 and HCC1143, and hemizygous deletions in the breast cancer cell line MDA-MB-134 and the peripheral blood cell lines HCC1143BL and Hs 578Bst obtained from the same breast cancer human subjects as cell lines HCC1143 and Hs 578T.

FIG. 21 shows graphical representations of DNA sequencing results showing a wild-type, a hemizygous mutant, and a heterozygous mutant of U50 as found in a normal (non-cancerous) cell sample, breast cancer cell line MDA-MB-134, and breast cell line MDA-MB-231, respectively.

FIG. 22 shows digital images of denaturing gel electrophoretic analyses of U50 homozygous mutations in two primary breast cancer samples compared to the matched normal cells in which U50 harbored hemizygous mutations.

FIG. 23 shows digital images of denaturing gel electrophoretic analyses of U50 homozygous mutations in two primary breast cancer samples compared to the matched normal cells in which U50 harbored only wild-type.

FIG. 24 shows digital images of denaturing gel electrophoretic analyses of U50 hemizygous deletion of U50 in three breast cancer subjects both in cancer (T) cells and matched normal (N) cells.

FIG. 25 is a graph illustrating the evaluation of U50 expression in transfected cells for colony formation assays.

FIG. 26 is a digital image showing U50-expression-reduced colony number in MDA-MB-231 cells.

FIG. 27 is a graph illustrating cell numbers estimated by the SRB staining and the measurement of optical densities (y axis) after U50 transfection into MDA-MB-231 and Hs 578T cells. *, P<0.005; **, P=0.009.

FIG. 28 is a graph illustrating cell numbers estimated by the SRB staining and the measurement of optical densities (y axis) after FOXO1A transfection into MDA-MB-231 and Hs 578T cells as positive control of colony formation assay. *, P<0.005; **, P<0.005.

FIG. 29 shows a digital image of a denaturing gel electrophoretic analysis showing both wild-type and mutant alleles expressed in samples in which the U50 genome showed heterozygosity.

FIG. 30 shows the nucleotide sequences according to SEQ ID NOs: 3-19.

The drawings are described in greater detail in the description and examples below.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DESCRIPTION OF THE DISCLOSURE

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. Patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### Definitions

The term “complementarity” or “complementary” as used herein refers to a sufficient number in the oligonucleotide of complementary base pairs in its sequence to interact specifically (hybridize) with the target nucleic acid sequence to be amplified or detected. As known to those skilled in the art, a very high degree of complementarity is needed for specificity and sensitivity involving hybridization, although it need not be 100%. Thus, for example, an oligonucleotide that is identical in nucleotide sequence to an oligonucleotide disclosed herein, except for one base change or substitution, may function equivalently to the disclosed oligonucleotides. A “complementary DNA” or “cDNA” gene includes recombinant genes synthesized by reverse transcription of messenger RNA (“mRNA”).

The term “cyclic polymerase-mediated reaction” as used herein refers to a biochemical reaction in which a template molecule or a population of template molecules is periodically and repeatedly copied to create a complementary template molecule or complementary template molecules, thereby increasing the number of the template molecules over time.

The term “denaturation” as used herein refers to the unfolding or other alteration of the structure of a template so as to make the template accessible to duplication. In the case of DNA, “denaturation” refers to the separation of the two complementary strands of the double helix, thereby creating two complementary, single stranded template molecules. “Denaturation” can be accomplished in any of a variety of ways, including by heat or by treatment of the DNA with a base or other denaturant.

The term “detectable amount of product” as used herein refers to an amount of amplified nucleic acid that can be detected using standard laboratory tools. A “detectable marker” refers to a nucleotide analog that allows detection using visual or other means. For example, fluorescently labeled nucleotides can be incorporated into a nucleic acid during one or more steps of a cyclic polymerase-mediated reaction, thereby allowing the detection of the product of the reaction using, e.g. fluorescence microscopy or other fluorescence-detection instrumentation.

The term “detectable moiety” as used herein refers to a label molecule (isotopic or non-isotopic) which is incorporated indirectly or directly into an oligonucleotide, wherein the label molecule facilitates the detection of the oligonucleotide in which it is incorporated. Thus, “detectable moiety” is used synonymously with “label molecule”. Synthesis of oligonucleotides can be accomplished by any one of several methods known to those skilled in the art. Label molecules, known to those skilled in the art as being useful for detection, include chemiluminescent or fluorescent molecules. Various fluorescent molecules are known in the art which are suitable for use to label a nucleic acid for the method of the present disclosure. The protocol for such incorporation may vary depending upon the fluorescent molecule used. Such protocols are known in the art for the respective fluorescent molecule.

By “detectably labeled” is meant that a fragment or an oligonucleotide contains a nucleotide that is radioactive, or that is substituted with a fluorophore, or that is substituted with some other molecular species that elicits a physical or chemical response that can be observed or detected by the naked eye or by means of instrumentation such as, without limitation, scintillation counters, colorimeters, UV spectrophotometers and the like.

The term “label” or “tag” as used herein may refer to a molecule that, when appended by, for example, without limitation, covalent bonding or hybridization, to another molecule, for example, also without limitation, a polynucleotide or polynucleotide fragment, provides or enhances a means of detecting the other molecule. A fluorescence or fluorescent label or tag emits detectable light at a particular wavelength when excited at a different wavelength. A radiolabel or radioactive tag emits radioactive particles detectable with an instrument such as, without limitation, a scintillation counter. Other signal generation detection methods include: chemiluminescence, electrochemiluminescence, raman, calorimetric, hybridization protection assay, and mass spectrometry

The term “DNA amplification” as used herein refers to any process that increases the number of copies of a specific DNA sequence by enzymatically amplifying the nucleic acid sequence. A variety of processes are known. One of the most commonly used is the polymerase chain reaction (PCR), which is defined and described in later sections below. The PCR process of Mullis is described in U.S. Pat. Nos. 4,683,195 and 4,683,202. PCR involves the use of a thermostable DNA polymerase, known sequences as primers, and heating cycles, which separate the replicating deoxyribonucleic acid (DNA), strands and exponentially amplify a gene of interest. Any type of PCR, such as quantitative PCR, RT-PCR, hot start PCR, LAPCR, multiplex PCR, touchdown PCR, etc., may be used. Advantageously, real-time PCR is used. In general, the PCR amplification process involves an enzymatic chain reaction for preparing exponential quantities of a specific nucleic acid sequence. It requires a small amount of a sequence to initiate the chain reaction and oligonucleotide primers that will hybridize to the sequence. In PCR the primers are annealed to denatured nucleic acid followed by extension with an inducing agent (enzyme) and nucleotides. This results in newly synthesized extension products. Since these newly synthesized sequences become templates for the primers, repeated cycles of denaturing, primer annealing, and extension results in exponential accumulation of the specific sequence being amplified. The extension product of the chain reaction will be a discrete nucleic acid duplex with a termini corresponding to the ends of the specific primers employed.

The term “DNA” as used herein refers to the polymeric form of deoxyribonucleotides (adenine, guanine, thymine, or cytosine) in either single stranded form, or as a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA).

The terms “enzymatically amplify” or “amplify” as used herein refer to DNA amplification, i.e., a process by which nucleic acid sequences are amplified in number. There are several means for enzymatically amplifying nucleic acid sequences. Currently the most commonly used method is the polymerase chain reaction (PCR). Other amplification methods include LCR (ligase chain reaction) which utilizes DNA ligase, and a probe consisting of two halves of a DNA segment that is complementary to the sequence of the DNA to be amplified, enzyme QB replicase and a ribonucleic acid (RNA) sequence template attached to a probe complementary to the DNA to be copied which is used to make a DNA template for exponential production of complementary RNA; strand displacement amplification (SDA); Qβ replicase amplification (QβRA); self-sustained replication (3SR); and NASBA (nucleic acid sequence-based amplification), which can be performed on RNA or DNA as the nucleic acid sequence to be amplified.

The term “fragment” of a molecule such as a protein or nucleic acid as used herein refers to any portion of the amino acid or nucleotide genetic sequence.

The term “genome” as used herein refers to all the genetic material in the chromosomes of a particular organism. Its size is generally given as its total number of base pairs. Within the genome, the term “gene” refers to an ordered sequence of nucleotides located in a particular position on a particular chromosome that encodes a specific functional product (e.g., a protein or RNA molecule). In general, a subject animal's or human's genetic characteristics, as defined by the nucleotide sequence of its genome, are known as its “genotype,” while the human subject's physical traits are described as its “phenotype.”

The term “heterozygous” or “heterozygous polymorphism” as used herein refers to the two alleles of a diploid cell or organism at a given locus are different, that is, that they have a different nucleotide exchanged for the same nucleotide at the same place in their sequences.

The term “homozygous” or “homozygous polymorphism” as used herein refers to the two alleles of a diploid cell or organism at a given locus are identical, that is, that they have the same nucleotide for nucleotide exchange at the same place in their sequences.

The term “hybridization” or “hybridizing,” as used herein refers to the formation of A-T and C-G base pairs between the nucleotide sequence of a fragment of a segment of a polynucleotide and a complementary nucleotide sequence of an oligonucleotide. By complementary is meant that at the locus of each A, C, G or T (or U in a ribonucleotide) in the fragment sequence, the oligonucleotide sequenced has a T, G, C or A, respectively. The hybridized fragment/oligonucleotide is called a “duplex.”

The term “hybridization complex”, such as in a sandwich assay, as used herein refers to a complex of nucleic acid molecules including at least the target nucleic acid and a sensor probe. It may also include an anchor probe.

The term “hybridizing under stringent conditions” as used herein refers to annealing a first nucleic acid to a second nucleic acid under stringent conditions as defined below. Stringent hybridization conditions typically permit the hybridization of nucleic acid molecules having at least 70% nucleic acid sequence identity with the nucleic acid molecule being used as a probe in the hybridization reaction. For example, the first nucleic acid may be a test sample or probe, and the second nucleic acid may be the sense or antisense strand of an ovomucoid gene expression control region or a fragment thereof. Hybridization of the first and second nucleic acids may be conducted under stringent conditions, e.g., high temperature and/or low salt content that tend to disfavor hybridization of dissimilar nucleotide sequences. Alternatively, hybridization of the first and second nucleic acid may be conducted under reduced stringency conditions, e.g. low temperature and/or high salt content that tend to favor hybridization of dissimilar nucleotide sequences. Low stringency hybridization conditions may be followed by high stringency conditions or intermediate medium stringency conditions to increase the selectivity of the binding of the first and second nucleic acids. The hybridization conditions may further include reagents such as, but not limited to, dimethyl sulfoxide (DMSO) or formamide to disfavor still further the hybridization of dissimilar nucleotide sequences. A suitable hybridization protocol may, for example, involve hybridization in 6×SSC (wherein 1×SSC comprises 0.015 M sodium citrate and 0.15 M sodium chloride), at 65° C. in an aqueous solution, followed by washing with 1×SSC at 65° C. Formulae to calculate appropriate hybridization and wash conditions to achieve hybridization permitting 30% or less mismatch between two nucleic acid molecules are disclosed, for example, in Meinkoth et al., (1984) Anal. Biochem. 138:267-284; the contents of which is incorporated herein by reference in its entirety. Protocols for hybridization techniques are well known to those of skill in the art and standard molecular biology manuals may be consulted to select a suitable hybridization protocol without undue experimentation. See, for example, Sambrook et al., 1989, “Molecular Cloning: A Laboratory Manual,” 2nd ed., Cold Spring Harbor Press: the contents of which is incorporated herein by reference in its entirety.

Typically, stringent conditions will be those in which the salt concentration is less than about 1.5 M Na ion, typically about 0.01 to 1.0 M Na ion concentration (or other salts) from about pH 7.0 to about pH 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

The terms “unique nucleic acid region” and “unique protein (polypeptide) region” as used herein refer to sequences present in a nucleic acid or protein (polypeptide) respectively that is not present in any other nucleic acid or protein sequence. The terms “conserved nucleic acid region” as referred to herein is a nucleotide sequence present in two or more nucleic acid sequences, to which a particular nucleic acid sequence can hybridize under low, medium or high stringency conditions. The greater the degree of conservation between the conserved regions of two or more nucleic acid sequences, the higher the hybridization stringency that will allow hybridization between the conserved region and a particular nucleic acid sequence.

The term “immobilized on a solid support” as used herein refers to a fragment, primer or oligonucleotide when attached to a substance at a particular location in such a manner that the system containing the immobilized fragment, primer or oligonucleotide may be subjected to washing or other physical or chemical manipulation without being dislodged from that location. A number of solid supports and means of immobilizing nucleotide-containing molecules to them are known in the art; any of these supports and means may be used in the methods of this disclosure.

The term “locus” or “loci” as used herein refers to the site of a gene on a chromosome. A single allele from each locus is inherited from each parent. Each human subject's particular combination of alleles is referred to as its “genotype”. Where both alleles are identical, the individual is said to be homozygous for the trait controlled by that pair of alleles; where the alleles are different, the individual is said to be heterozygous for the trait.

The term “melting temperature” as used herein refers to the temperature at which hybridized duplexes dehybridize and return to their single-stranded state. Likewise, hybridization will not occur in the first place between two oligonucleotides, or, herein, an oligonucleotide and a fragment, at temperatures above the melting temperature of the resulting duplex. It is presently advantageous that the difference in melting point temperatures of oligonucleotide-fragment duplexes of this disclosure be from about 1° C. to about 10° C. so as to be readily detectable.

The term “nucleic acid molecule” as used herein refers to DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but advantageously is double-stranded DNA. An “isolated” nucleic acid molecule is one that is separated from other nucleic acid molecules that are present in the natural source of the nucleic acid. A “nucleoside” refers to a base linked to a sugar. The base may be adenine (A), guanine (G) (or its substitute, inosine (I)), cytosine (C), or thymine (T) (or its substitute, uracil (U)). The sugar may be ribose (the sugar of a natural nucleotide in RNA) or 2-deoxyribose (the sugar of a natural nucleotide in DNA). A “nucleotide” refers to a nucleoside linked to a single phosphate group.

The term “oligonucleotide” as used herein refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides may be chemically synthesized and may be used as primers or probes. Oligonucleotide means any nucleotide of more than 3 bases in length used to facilitate detection or identification of a target nucleic acid, including probes and primers.

The term “polymerase chain reaction” or “PCR” as used herein refers to a thermocyclic, polymerase-mediated, DNA amplification reaction. A PCR typically includes template molecules, oligonucleotide primers complementary to each strand of the template molecules, a thermostable DNA polymerase, and deoxyribonucleotides, and involves three distinct processes that are multiply repeated to effect the amplification of the original nucleic acid. The three processes (denaturation, hybridization, and primer extension) are often performed at distinct temperatures, and in distinct temporal steps. In many embodiments, however, the hybridization and primer extension processes can be performed concurrently. The nucleotide sample to be analyzed may be PCR amplification products provided using the rapid cycling techniques described in U.S. Pat. Nos. 6,569,672; 6,569,627; 6,562,298; 6,556,940; 6,569,672; 6,569,627; 6,562,298; 6,556,940; 6,489,112; 6,482,615; 6,472,156; 6,413,766; 6,387,621; 6,300,124; 6,270,723; 6,245,514; 6,232,079; 6,228,634; 6,218,193; 6,210,882; 6,197,520; 6,174,670; 6,132,996; 6,126,899; 6,124,138; 6,074,868; 6,036,923; 5,985,651; 5,958,763; 5,942,432; 5,935,522; 5,897,842; 5,882,918; 5,840,573; 5,795,784; 5,795,547; 5,785,926; 5,783,439; 5,736,106; 5,720,923; 5,720,406; 5,675,700; 5,616,301; 5,576,218 and 5,455,175, the disclosures of which are incorporated by reference in their entireties. Other methods of amplification include, without limitation, NASBR, SDA, 3SR, TSA and rolling circle replication. It is understood that, in any method for producing a polynucleotide containing given modified nucleotides, one or several polymerases or amplification methods may be used. The selection of optimal polymerization conditions depends on the application.

The term “polymerase” as used herein refers to an enzyme that catalyzes the sequential addition of monomeric units to a polymeric chain, or links two or more monomeric units to initiate a polymeric chain. In advantageous embodiments of this disclosure, the “polymerase” will work by adding monomeric units whose identity is determined by and which is complementary to a template molecule of a specific sequence. For example, DNA polymerases such as DNA pol 1 and Taq polymerase add deoxyribonucleotides to the 3′ end of a polynucleotide chain in a template-dependent manner, thereby synthesizing a nucleic acid that is complementary to the template molecule. Polymerases may be used either to extend a primer once or repetitively or to amplify a polynucleotide by repetitive priming of two complementary strands using two primers.

The term “polynucleotide” as used herein refers to a linear chain of nucleotides connected by a phosphodiester linkage between the 3′-hydroxyl group of one nucleoside and the 5′-hydroxyl group of a second nucleoside which in turn is linked through its 3′-hydroxyl group to the 5′-hydroxyl group of a third nucleoside and so on to form a polymer comprised of nucleosides liked by a phosphodiester backbone. A “modified polynucleotide” refers to a polynucleotide in which one or more natural nucleotides have been partially or substantially replaced with modified nucleotides.

The term “primer” as used herein refers to an oligonucleotide, the sequence of at least a portion of which is complementary to a segment of a template DNA which to be amplified or replicated. Typically primers are used in performing the polymerase chain reaction (PCR). A primer hybridizes with (or “anneals” to) the template DNA and is used by the polymerase enzyme as the starting point for the replication/amplification process. By “complementary” is meant that the nucleotide sequence of a primer is such that the primer can form a stable hydrogen bond complex with the template; i.e., the primer can hybridize or anneal to the template by virtue of the formation of base-pairs over a length of at least ten consecutive base pairs.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

The term “probes” as used herein refers to oligonucleotide nucleic acid sequences of variable length, used in the detection of identical, similar, or complementary nucleic acid sequences by hybridization. An oligonucleotide sequence used as a detection probe may be labeled with a detectable moiety. Various labeling moieties are known in the art. Said moiety may, for example, either be a radioactive compound, a detectable enzyme (e.g. horse radish peroxidase (HRP)) or any other moiety capable of generating a detectable signal such as a calorimetric, fluorescent, chemiluminescent or electrochemiluminescent signal. The detectable moiety may be detected using known methods.

The term “protein” as used herein refers to a large molecule composed of one or more chains of amino acids in a specific order. The order is determined by the base sequence of nucleotides in the gene coding for the protein. Proteins are required for the structure, function, and regulation of the body's cells, tissues, and organs. Each protein has a unique function.

The term “restriction enzyme” as used herein refers to an endonuclease (an enzyme that cleaves phosphodiester bonds within a polynucleotide chain) that cleaves DNA in response to a recognition site on the DNA. The recognition site (restriction site) may be a specific sequence of nucleotides typically about 4-8 nucleotides long.

The term “template” as used herein refers to a target polynucleotide strand, for example, without limitation, an unmodified naturally-occurring DNA strand, which a polymerase uses as a means of recognizing which nucleotide it should next incorporate into a growing strand to polymerize the complement of the naturally-occurring strand. Such DNA strand may be single-stranded or it may be part of a double-stranded DNA template. In applications of the present disclosure requiring repeated cycles of polymerization, e.g., the polymerase chain reaction (PCR), the template strand itself may become modified by incorporation of modified nucleotides, yet still serve as a template for a polymerase to synthesize additional polynucleotides.

The term “thermocyclic reaction” as used herein refers to a multi-step reaction wherein at least two steps are accomplished by changing the temperature of the reaction.

The term “thermostable polymerase” as used herein refers to a DNA or RNA polymerase enzyme that can withstand extremely high temperatures, such as those approaching 100° C. Often, thermostable polymerases are derived from organisms that live in extreme temperatures, such as Thermus aquaticus. Examples of thermostable polymerases include Taq, Tth, Pfu, Vent, deep vent, UlTma, and variations and derivatives thereof.

Unless otherwise defined, all technical and scientific terms used herein have the same meaning as commonly understood by one of ordinary skill in the art of molecular biology. Although methods and materials similar or equivalent to those described herein can be used in the practice or testing of the present disclosure, suitable methods and materials are described herein.

Further definitions are provided in context below. Unless otherwise defined, all technical and scientific terms used herein have the same meaning as commonly understood by one of ordinary skill in the art of molecular biology. Although methods and materials similar or equivalent to those described herein can be used in the practice or testing of the present disclosure, suitable methods and materials are described herein.

### Description

Genetic and functional analyses were performed and it was discovered that the U50 snoRNA gene, encoded by an intron, is a 6q tumor-suppressor gene. It was also discovered that a 2 bp germline homozygous deletion of U50 is associated with clinically significant prostate and breast cancers in large cohorts.

Deletion of chromosome 6q14-q22 is common in multiple human cancers including prostate cancer and breast, and chromosome 6 transferred into cancer cells induces senescence and reduces cell growth, tumorigenicity and metastasis, indicating the existence of one or more tumor-suppressor genes in 6q. To identify the 6q tumor-suppressor gene, the common region of deletion was first narrowed to a 2.5 Mb interval at 6q14-15. Of the 11 genes located in this minimal deletion region and expressed in normal prostate and breast tissues, only snoRNA U50 was mutated, demonstrated transcriptional down-regulation, and inhibited colony formation in prostate and breast cancer cells. The mutation, a homozygous 2 bp (TT) deletion, was found in two of 30 prostate cancer cell lines/xenografts and nine of 89 localized prostate cancers (eleven of 119 or 9% cancers). Two of 89 (2%) human subjects with prostate cancer also showed the same mutation in their germline DNA, but none of 104 cancer-free control men did. The homozygous deletion abolished U50 function in a colony formation assay. Analysis of 1371 prostate cancer cases and 1371 matched control men from a case-control study nested in a prospective cohort showed that although a germline heterozygous genotype of the deletion was detected in both human subjects and controls at similar frequencies, the homozygosity of the deletion was significantly associated with clinically significant prostate cancer (odds ratio 2.9; 95% confidence interval 1.17-7.21). snoRNA U50 is, therefore, established as a candidate for the 6q tumor-suppressor gene in prostate and breast cancer.

Similarly, although no homozygous deletions were found in 31′ breast cancer cell lines, 9 such lines showed transcription intensities less than half that of controls. Eight of the nine cell lines had heterozygous deletions with the U50 region deleted being identical to that deleted in prostate cancer cells.

### Tissue and DNA Samples

To determine the genotype of a subject human subject according to the methods of the present disclosure, it is necessary to obtain a sample of genomic DNA from that human subject. Typically, that sample of genomic DNA will be obtained from a sample of tissue or cells taken from that human subject.

A tissue or cell sample may be taken from a human subject at any time in the lifetime of the human subject for the determination of a germline polymorphism. The tissue sample can comprise hair (including roots), buccal swabs, blood, saliva, semen, muscle or from any internal organs. In the method of the present disclosure, the source of the tissue sample, and thus also the source of the test nucleic acid sample, is not critical. For example, the test nucleic acid can be obtained from cells within a body fluid of the human subject, or from cells constituting a body tissue of the human subject. The particular body fluid from which cells are obtained is also not critical to the present disclosure. For example, the body fluid may be selected from, but is not limited to, the group consisting of: blood, ascites, pleural fluid and spinal fluid.

The particular body tissue from which cells are obtained is also not critical to the present disclosure. For example, the body tissue can include, but is not limited to, skin, endometrial, uterine and cervical tissue. For the purposes of this disclosure, when a somatic mutation within the U50 locus is to be investigated, the tissue will be obtained from the prostate or breast of the human subject. Normal, tumor, or potentially tumorous tissues can be isolated from the prostate or breast at the same time or in the same biopsy sample. The tumorous and non-tumorous cells within the sample may be isolated therefrom for subsequent analysis of the U50 polymorphism. Whatever source of cells or tissue is used, a sufficient amount of cells must be obtained to provide a sufficient amount of DNA for analysis. This amount will be known or readily determinable by those skilled in the art.

DNA may be isolated from the tissue/cells by techniques known to those skilled in the art (see, e.g., U.S. Pat. Nos. 6,548,256 and 5,989,431, Hirota et al., (1989) Jinrui Idengaku Zasshi. 34:217-23 and John et al., (1991) Nucleic Acids Res. 19:408; the disclosures of which are incorporated by reference in their entireties). For example, high molecular weight DNA may be purified from cells or tissue using proteinase K extraction and ethanol precipitation. DNA may be extracted from a human subject specimen using any other suitable methods known in the art.

### Determining the Genotype of an Human Subject of Interest

The present disclosure provides methods for determining the genotype of a given human subject to identify human subjects carrying specific alleles of the U50 locus, and in particular a TT deletion compared to a control sequence, and use of the genotype as a predictive prognostic tool to determine the presence or outcome of a prostate or breast cancer. There are many methods known in the art for determining the genotype of a human subject and for identifying whether a given DNA sample contains a particular polymorphism. Any method for determining genotype can be used for determining the genotype in the present disclosure. Such methods include, but are not limited to, amplimer sequencing, DNA sequencing, fluorescence spectroscopy, fluorescence resonance energy transfer (or “FRET”)-based hybridization analysis, high throughput screening, mass spectroscopy, nucleic acid hybridization, polymerase chain reaction (PCR), RFLP analysis and size chromatography (e.g., capillary or gel chromatography), all of which are well known to one of skill in the art. In particular, methods for determining nucleotide polymorphisms, particularly single nucleotide polymorphisms, are described in U.S. Pat. Nos. 6,514,700; 6,503,710; 6,468,742; 6,448,407; 6,410,231; 6,383,756; 6,358,679; 6,322,980; 6,316,230; and 6,287,766 and reviewed by Chen & Sullivan, Pharmacogenomics J. (2003) 3:77-96, the disclosures of which are incorporated by reference in their entireties.

### Determining the Genotype by Sequencing

In one embodiment, the presence or absence of the TT deletion of U50 is determined by sequencing the region of the genomic DNA sample that spans the polymorphic locus. Many methods of sequencing genomic DNA are known in the art, and any such method can be used, see for example Sambrook et al., Molecular Cloning; A Laboratory Manual 2d ed. (1989). For example, as described below, a DNA fragment spanning the location of the polymorphism of interest can be amplified using the polymerase chain reaction or some other cyclic polymerase mediated amplification reaction. The amplified region of DNA can then be sequenced using any method known in the art. Advantageously, the nucleic acid sequencing is by automated methods (reviewed by Meldrum, (2000) Genome Res. 10:1288-303, the disclosure of which is incorporated by reference in its entirety), for example using a Beckman CEQ 8000 Genetic Analysis System (Beckman Coulter Instruments, Inc.). Methods for sequencing nucleic acids include, but are not limited to, automated fluorescent DNA sequencing (see, e.g., Watts & MacBeath, (2001) Methods Mol. Biol. 167:153-170; and MacBeath et al., (2001) Methods Mol. Biol. 167:119-152), capillary electrophoresis (see, e.g., Bosserhoff et al., (2000) Comb. Chem. High Throughput Screen 3: 455-466), DNA sequencing chips (see, e.g., Jain, (2000) Pharmacogenomics. 1:289-307), mass spectrometry (see, e.g., Yates, (2000) Trends Genet. 16:5-8), pyrosequencing (see, e.g., Ronaghi, (2001) Genome Res. 11:3-11), and ultrathin-layer gel electrophoresis (see, e.g., Guttman & Ronai, (2000) Electrophoresis. 21:3952-64), the disclosures of which are hereby incorporated by reference in their entireties. The sequencing can also be done by a commercial company. Examples of such companies include, but are not limited to, the University of Georgia Molecular Genetics Instrumentation Facility (Athens, Ga.) or SeqWright DNA Technologies Services (Houston, Tex.).

### Determining the Genotype Using Cyclic Polymerase Mediated Amplification

In certain embodiments of the present disclosure, the detection of a given single nucleotide polymorphism (SNP) can be performed using cyclic polymerase-mediated amplification methods. Any one of the methods known in the art for amplification of DNA may be used, such as for example, the polymerase chain reaction (PCR), the ligase chain reaction (LCR) (Barany, (1991) Proc. Natl. Acad. Sci. (U.S.A.) 88:189-193), the strand displacement assay (SDA), or the oligonucleotide ligation assay (“OLA”) (Landegren et al., (1988) Science 241:1077-1080). Nickerson et al. have described a nucleic acid detection assay that combines attributes of PCR and OLA (Nickerson et al., (1990) Proc. Natl. Acad. Sci. (U.S.A.) 87:8923-8927). Other known nucleic acid amplification procedures, such as transcription-based amplification systems (Malek et al., U.S. Pat. No. 5,130,238; Davey et al., European Patent Application 329,822; Schuster et al., U.S. Pat. No. 5,169,766; Miller et al., PCT Application WO89/06700; Kwoh et al., (1989) Proc. Natl. Acad. Sci. (U.S.A.) 86:1173; Gingeras et al., PCT Application WO88/10315)), or isothermal amplification methods (Walker et al., (1992) Proc. Natl. Acad. Sci. (U.S.A.) 89:392-396) may also be used.

The most advantageous method of amplifying DNA fragments containing the polymorphisms or mutations of the disclosure employs PCR (see e.g., U.S. Pat. Nos. 4,965,188; 5,066,584; 5,338,671; 5,348,853; 5,364,790; 5,374,553; 5,403,707; 5,405,774; 5,418,149; 5,451,512; 5,470,724; 5,487,993; 5,523,225; 5,527,510; 5,567,583; 5,567,809; 5,587,287; 5,597,910; 5,602,011; 5,622,820; 5,658,764; 5,674,679; 5,674,738; 5,681,741; 5,702,901; 5,710,381; 5,733,751; 5,741,640; 5,741,676; 5,753,467; 5,756,285; 5,776,686; 5,811,295; 5,817,797; 5,827,657; 5,869,249; 5,935,522; 6,001,645; 6,015,534; 6,015,666; 6,033,854; 6,043,028; 6,077,664; 6,090,553; 6,168,918; 6,174,668; 6,174,670; 6,200,747; 6,225,093; 6,232,079; 6,261,431; 6,287,769; 6,306,593; 6,440,668; 6,468,743; 6,485,909; 6,511,805; 6,544,782; 6,566,067; 6,569,627; 6,613,560; 6,613,560 and 6,632,645; the disclosures of which are incorporated by reference in their entireties), using primer pairs that are capable of hybridizing to the proximal sequences that define or flank a polymorphic site in its double-stranded form.

To perform a cyclic polymerase-mediated amplification reaction according to the present disclosure, the primers are hybridized or annealed to opposite strands of the target DNA, the temperature is then raised to permit the thermostable DNA polymerase to extend the primers and thus replicate the specific segment of DNA spanning the region between the two primers. Then the reaction is thermocycled so that at each cycle the amount of DNA representing the sequences between the two primers is doubled, and specific amplification of the DNA sequences, if present, results.

Any of a variety of polymerases can be used in the present disclosure. For thermocyclic reactions, the polymerases are thermostable polymerases such as Taq, KlenTaq, Stoffel Fragment, Deep Vent, Tth, Pfu, Vent, and UlTma, each of which are readily available from commercial sources. For non-thermocyclic reactions, and in certain thermocyclic reactions, the polymerase will often be one of many polymerases commonly used in the field and commercially available such as DNA pol 1, Klenow fragment, T7 DNA polymerase, and T4 DNA polymerase, and the like. Guidance for the use of such polymerases can readily be found in product literature and in general molecular biology guides.

Typically, the annealing of the primers to the target DNA sequence is carried out for about 2 minutes at about 37-55° C., extension of the primer sequence by the polymerase enzyme (such as Taq polymerase) in the presence of nucleoside triphosphates is carried out for about 3 minutes at about 70-75° C., and the denaturing step to release the extended primer is carried out for about 1 minute at about 90-95° C. However, these parameters can be varied, and one of skill in the art would readily know how to adjust the temperature and time parameters of the reaction to achieve the desired results. For example, cycles may be as short as 10, 8, 6, 5, 4.5, 4, 2, 1, 0.5 minutes or less.

Also, “two temperature” techniques can be used where the annealing and extension steps may both be carried out at the same temperature, typically between about 60-65° C., thus reducing the length of each amplification cycle and resulting in a shorter assay time.

Typically, the reactions described herein are repeated until a detectable amount of product is generated. Often, such detectable amounts of product are between about 10 ng and about 100 ng, although larger quantities, e.g. 200 ng, 500 ng, 1 μg or more can also, of course, be detected. In terms of concentration, the amount of detectable product can be from about 0.01 pmol, 0.1 pmol, 1 pmol, 10 pmol, or more. Thus, the number of cycles of the reaction that are performed can be varied, the more cycles are performed, the more amplified product is produced. In certain embodiments, the reaction comprises 2, 5,10,15, 20, 30, 40, 50, or more cycles.

For example, the PCR reaction may be carried out using about 25-50 μl samples containing about 0.01 to 1.0 ng of template amplification sequence, about 10 to 100 pmol of each generic primer, about 1.5 units of Taq DNA polymerase (Promega Corp.), about 0.2 mM dDATP, about 0.2 mM dCTP, about 0.2 mM dGTP, about 0.2 mM dTTP, about 15 mM MgCl₂, about 10 mM Tris-HCl (pH 9.0), about 50 mM KCl, about 1 μg/ml gelatin, and about 10 μl/ml Triton X-100 (Saiki, 1988).

Those of skill in the art are aware of the variety of nucleotides available for use in the cyclic polymerase mediated reactions. Typically, the nucleotides can be at least in part of deoxynucleotide triphosphates (dNTPs), which are readily commercially available. Parameters for optimal use of dNTPs are also known to those of skill, and are described in the literature. In addition, a large number of nucleotide derivatives are known to those of skill and can be used in the present reaction. Such derivatives include fluorescently labeled nucleotides, allowing the detection of the product including such labeled nucleotides, as described below. Also included in this group are nucleotides that allow the sequencing of nucleic acids including such nucleotides, such as chain-terminating nucleotides, dideoxynucleotides and boronated nuclease-resistant nucleotides. Commercial kits containing the reagents most typically used for these methods of DNA sequencing are available and widely used. Other nucleotide analogs include nucleotides with bromo-, iodo-, or other modifying groups, which affect numerous properties of resulting nucleic acids including their antigenicity, their replicatability, their melting temperatures, their binding properties, etc. In addition, certain nucleotides include reactive side groups, such as sulfhydryl groups, amino groups, N-hydroxysuccinimidyl groups, that allow the further modification of nucleic acids comprising them.

The present disclosure provides oligonucleotides that can be used as primers to amplify the U50-specific nucleic acid sequence. In certain embodiments, these primers can be oligonucleotide fragments. Such fragments should be of sufficient length to enable specific annealing or hybridization to the nucleic acid sample. The sequences typically will be about 8 to about 44 nucleotides in length, but may be longer. Longer sequences, e.g., from about 14 to about 50, are advantageous for certain embodiments.

In embodiments where it is desired to amplify a fragment of DNA comprising the U50 locus, primers having contiguous stretches of about 8, 9, 10, 11, 12, 13, 14, 15, 16, 17, 18, 19, 20, 21, 22, 23, or 24 nucleotides, as described by Dong et al ((2008) Hum. Mol. Genet. 17:1031-1042, incorporated herein by reference in its entirety, and derived from a genomic nucleotide sequence such as, for example, that of GenBank Accession No. AB017710 as disclosed by Tanaka et al., (2000) Genes Cells 5:277-287, incorporated herein by reference in its entirety, are contemplated.

Although various different lengths of primers can be used, and the exact location of the stretch of contiguous nucleotides in U50 gene used to make the primer can vary, it is important that the sequences to which the forward and reverse primers anneal are located on either side of the particular nucleotide positions that my be deleted in polymorphic variants of the U50 locus. For example, when designing primers for amplification of the ΔTT polymorphism of U50, one primer must be located upstream of (but not overlapping with) the nucleotide positions 54,55 of the snoRNA U50-encoding sequence (SEQ ID NO: 1), and the other primer must be located downstream of (but not overlapping with) nucleotide positions 54 and 55 of the sequence SEQ ID NO: 1.

In a preferred embodiment, a fragment of DNA spanning and containing the location of the ΔTTU50 polymorphism, i.e. at least a region that includes the nucleotides from nucleotide position 47 to position 60 of the nucleotide sequence according to SEQ ID NO: 1, may be amplified from a nucleic acid sample template using a primer having the sequence:

(SEQ ID NO: 3)

5′-TCGAGCGGCCGCCCGGGCAGGTATCTCAGAAGCCAGATCCG-3′,

and a primer having the sequence:

| | |
|---|---|
| 5′-TTCTGTGATGATCTTATCCCGAACCTGAAC-3′ | (SEQ ID NO: 4) |
| or | |
| 5′-ATCTCAGAAGCCAGATCCGTAAAAG-3′ | (SEQ ID NO:7) |

The above methods employ primers located on each side of, and not overlapping with, the ΔTTU50 polymorphism to amplify a fragment of DNA that includes the nucleotide position at which the polymorphism is located. Such methods require additional steps such as sequencing of the fragment, or hybridization of allele specific probes to the fragment, to determine the genotype at the polymorphic site. However, in some embodiments of the present disclosure, the amplification method is itself a method for determining the genotype of the polymorphic site, as for example, in “allele-specific PCR”. In allele-specific PCR, primer pairs are chosen such that amplification itself is dependent upon the input template nucleic acid containing the polymorphism of interest. In such embodiments, primer pairs are chosen such that at least one primer spans the actual nucleotide position of the polymorphism and is therefore an allele-specific oligonucleotide primer. Typically, a primer contains a single allele-specific nucleotide at the 3′ terminus preceded by bases that are complementary to the gene of interest. The PCR reaction conditions are adjusted such that amplification by a DNA polymerase proceeds from matched 3′-primer termini, but does not proceed where a mismatch occurs. Allele-specific PCR can be performed in the presence of two different allele-specific primers, one specific for each allele, where each primer is labeled with a different dye, for example one allele specific primer may be labeled with a green dye (e.g. fluorescein) and the other allele specific primer labeled with a red dye (e.g. sulforhodamine). Following amplification, the products are analyzed for green and red fluorescence. The aim is for one homozygous genotype to yield green fluorescence only, the other homozygous genotype to give red fluorescence only, and the heterozygous genotype to give mixed red and green fluorescence.

Thus, to perform allele specific PCR to detect the ΔTTU50 polymorphism, one primer must overlap nucleotide positions 54 and 55 of SEQ ID NO: 1 such that nucleotide positions 54 and 55 are at the 3′ terminus of the primer. Suitable primers are disclosed herein in Example 3, below.

Methods for performing allele specific PCR are well known in the art, and any such methods may be used. For example suitable methods are taught in Myakishev et al., (2001) Genome Research 1:163-169; Alexander et al., (2004) Mol. Biotechnol. 28:171-174; and Ruano et al. (1989) Nucleic Acids Res. 17:8392, the contents of which are incorporated by reference. To perform, allele specific PCR the reaction conditions must be carefully adjusted such that the allele specific primer will only bind to one allele and not the alternative allele, for example, in some embodiments the conditions are adjusted so that the primers will only bind where there is a 100% match between the primer sequence and the DNA, and will not bind if there is a single nucleotide mismatch.

### Determining the Genotype Using Hybridization-Based Methods

In certain embodiments of the present disclosure, the detection of the ΔTTU50 polymorphism can be performed using oligonucleotide probes that bind or hybridize to the DNA. The present disclosure, therefore, provides oligonucleotide probes that allow detection of the ΔTTU50 polymorphism in the human snoRNA, or the encoding gene.

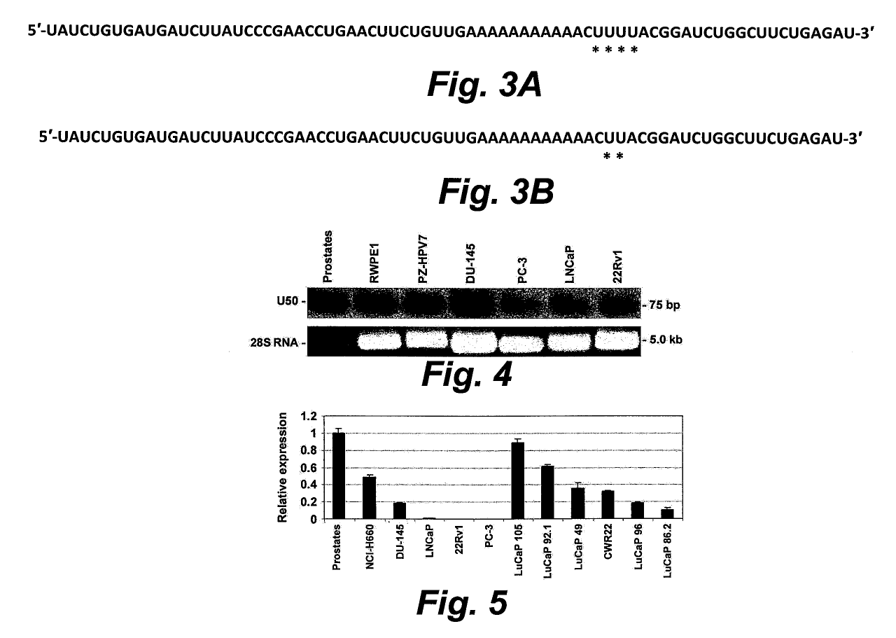

In certain embodiments, these probes may be oligonucleotide fragments. Such fragments should be of sufficient length to provide specific hybridization to the nucleic acid sample. The sequences typically will be about 8 to about 50 nucleotides, but may be longer. Nucleic acid probes may have contiguous stretches of about 8, 9, 10, 11, 12, 13, 14, 15, 16, 17, 18, 19, 20, 21, 22, 23, or 24 nucleotides from a sequence selected from SEQ ID NO: 1 (wild-type U50) as shown in FIG. 3A, or SEQ ID NO: 2 (ΔTTU50) as shown in FIG. 3B.

The probe sequence must span the particular nucleotide position that is deleted in the ΔTTU50 polymorphism to be detected. For example, probes designed for detection of the ΔTTU50 polymorphism must span nucleotide positions 54 and 55 of the U50 locus (SEQ ID NO: 1).

These probes will be useful in a variety of hybridization embodiments, such as Southern blotting, Northern blotting, and hybridization disruption analysis. Also the probes of the disclosure can be used to detect the ΔTTU50 polymorphism in amplified sequences, such as amplified PCR products generated using the primers described above. For example, in one embodiment a target nucleic acid is first amplified, such as by PCR or strand displacement amplification (SDA), and the amplified double stranded DNA product is then denatured and hybridized with a probe.

In other embodiments of the disclosure, double stranded DNA (amplified or not) may be denatured and hybridized with a probe of the present disclosure and then the hybridization complex may be subjected to destabilizing or disrupting conditions. By determining the level of disruption energy required wherein the probe has different disruption energy for one allele as compared to another allele, the genotype of a gene at a polymorphic locus can be determined. In one example, there can be lower disruption energy, e.g., melting temperature, for an allele that harbors a cytosine residue at a polymorphic locus, and a higher required energy for an allele with a thymine residue at that polymorphic locus. This can be achieved where the probe has 100% homology with one allele (a perfectly matched probe), but has a mismatch with the alternative allele e.g. the ΔTTU50 polymorphism. Since the perfectly matched probe is bound more tightly to the target DNA than the mismatched probe, it requires more energy to cause the hybridized probe to dissociate.

In one embodiment the destabilizing conditions comprise an elevation of temperature. The higher the temperature, the greater is the degree of destabilization. In another embodiment, the destabilizing conditions comprise subjecting the hybridization complex to a temperature gradient, whereby, as the temperature is increased, the degree of destabilization increases. In an alternative embodiment, the destabilizing conditions comprise treatment with a destabilizing compound, or a gradient comprising increasing amounts of such a compound. Suitable destabilizing compounds include, but are not limited to, salts and urea. Methods of destabilizing or denaturing hybridization complexes are well known in the art, and any such method may be used in accordance with the present disclosure. For example, methods of destabilizing or denaturing hybridization complexes are taught by Sambrook et al., Molecular Cloning; A Laboratory Manual 2d ed. (1989).

For optimal detection of single-base pair mismatches, it is preferable that there is about a 1° C. to about a 10° C. difference in melting temperature of the probe DNA complex when bound to one allele as opposed to the alternative allele at the polymorphic site. Thus, when the temperature is raised above the melting temperature of a probe-DNA duplex corresponding to one of the alleles, that probe will disassociate.

In other embodiments, two different “allele-specific probes” can be used for analysis of a single nucleotide polymorphism, a first allele-specific probe for detection of one allele, and a second allele-specific probe for the detection of the alternative allele. For example, in one embodiment the different alleles of the polymorphism can be detected using two different allele-specific probes, one for detecting the ΔTT-containing allele at nucleotide positions 54,55, and another for detecting the TT-containing allele (wild-type) at nucleotide position 54,55. In a preferred embodiment, an oligonucleotide probes may have, but are not limited to, the sequences:

(SEQ ID NO: 18; U50-specific)

5′-ATCTCAGAAGCCAGATCCG TAAAAG-3′
and

(SEQ ID NO: 19; U50ΔTT-specific)

5′-ATCTCAGAAGCCAGATC CGTAAG-3′.

Whichever probe sequences and hybridization methods are used, one skilled in the art can readily determine suitable hybridization conditions, such as temperature and chemical conditions. Such hybridization methods are well known in the art. For example, for applications requiring high selectivity, one will typically desire to employ relatively stringent conditions for the hybridization reactions, e.g., one will select relatively low salt and/or high temperature conditions, such as provided by about 0.02 M to about 0.10 M NaCl at temperatures of about 50° C. to about 70° C. Such high stringency conditions tolerate little, if any, mismatch between the probe and the template or target strand, and are particularly suitable for detecting specific SNPs according to the present disclosure. It is generally appreciated that conditions can be rendered more stringent by the addition of increasing amounts of formamide. Other variations in hybridization reaction conditions are well known in the art (see for example, Sambrook et al., Molecular Cloning; A Laboratory Manual 2d ed. (1989)).

### Producing the Primers and Probes of the Disclosure

The primers and probes described herein may be readily prepared by, for example, directly synthesizing the fragment by chemical means or by introducing selected sequences into recombinant vectors for recombinant production.

Defined oligonucleotides may be produced by any of several well known methods, including automated solid-phase chemical synthesis using cyanoethylphosphoramidite precursors. Barone et al., Nucleic Acids Research 12:4051 (1984). In addition, other well-known methods for construction of synthetic oligonucleotides may be employed.

Following synthesis and purification of an oligonucleotide, several different procedures may be utilized to determine the acceptability of the oligonucleotide in terms of size and purity. Such procedures include polyacrylamide gel electrophoresis and high pressure liquid chromatography, both of which are known to those skilled in the art.

Methods for making a vector or recombinants or plasmid for amplification of the fragment either in vivo or in vitro can be any desired method, e.g., a method which is by or analogous to the methods disclosed in, or disclosed in documents cited in: U.S. Pat. Nos. 4,603,112; 4,769,330; 4,394,448; 4,722,848; 4,745,051; 4,769,331; 4,945,050; 5,494,807; 5,514,375; 5,744,140; 5,744,141; 5,756,103; 5,762,938; 5,766,599; 5,990,091; 5,174,993; 5,505,941; 5,338,683; 5,494,807; 5,591,639; 5,589,466; 5,677,178; 5,591,439; 5,552,143; 5,580,859; 6,130,066; 6,004,777; 6,130,066; 6,497,883; 6,464,984; 6,451,770; 6,391,314; 6,387,376; 6,376,473; 6,368,603; 6,348,196; 6,306,400; 6,228,846; 6,221,362; 6,217,883; 6,207,166; 6,207,165; 6,159,477; 6,153,199; 6,090,393; 6,074,649; 6,045,803; 6,033,670; 6,485,729; 6,103,526; 6,224,882; 6,312,682; 6,348,450 and 6; 312,683; WO 90/01543; WO91/11525; WO 94/16716; WO 96/39491; WO 98/33510; EP 265785; EP 0 370 573; Andreansky et al., (1996) Proc. Natl. Acad. Sci. USA 93:11313-11318; Ballay et al., (1993) EMBO J. 4:3861-65; Feigner et al., (1994) J. Biol. Chem. 269:2550-2561; Frolov et al., (1996) Proc. Natl. Acad. Sci. USA 93:11371-11377; Graham, (1990) Tibtech;8:85-87; Grunhaus et al., (1992) Sem. Virol. 3:237-52; Ju et al., (1998) Diabetologia;41:736-739; Kitson et al., (1991) J. Virol. 65:3068-3075; McClements et al., (19960 Proc. Natl. Acad. Sci. USA 93:11414-11420; Moss (1996) Proc. Natl. Acad. Sci. USA 93:11341-11348; Paoletti (1996) Proc. Natl. Acad. Sci. USA 93:11349-11353.

### Labeling and Detecting the Primers and Probes of the Disclosure

Oligonucleotide sequences used as primers or probes according to the present disclosure may be labeled with a detectable moiety. As used herein, the term “sensors” refers to such primers or probes labeled with a detectable moiety. Various labeling moieties are known in the art. Said moiety may be, for example, a radiolabel (e.g., ³H, ¹²⁵I, ³⁵S, ¹⁴C, ³²P, etc.), detectable enzyme (e.g., horse radish peroxidase (HRP), alkaline phosphatase etc.), a fluorescent dye (e.g., fluorescein isothiocyanate, Texas red, rhodamine, Cy3, Cy5, Bodipy, Bodipy Far Red, Lucifer Yellow, Bodipy 630/650-X, Bodipy R6G-X and 5-CR 6G, and the like), a colorimetric label such as colloidal gold or colored glass or plastic (e.g., polystyrene, polypropylene, latex, etc.), beads, or any other moiety capable of generating a detectable signal such as a calorimetric, fluorescent, chemiluminescent or electrochemiluminescent (ECL) signal.

Primers or probes may be labeled directly or indirectly with a detectable moiety, or synthesized to incorporate the detectable moiety. In one embodiment, a detectable label is incorporated into a nucleic acid during at least one cycle of a cyclic polymerase-mediated amplification reaction. For example, polymerases can be used to incorporate fluorescent nucleotides during the course of polymerase-mediated amplification reactions. Alternatively, fluorescent nucleotides may be incorporated during synthesis of nucleic acid primers or probes. To label an oligonucleotide with the fluorescent dye, one of conventionally-known labeling methods can be used ((1996) Nature Biotech. 14:303-308; (1997) Applied Environmental Microbiol. 63:1143-1147; (1996) Nucleic Acids Res. 24:4532-4535). An advantageous probe is one labeled with a fluorescent dye at the 3′ or 5′ end and containing G or C as the base at the labeled end. If the 5′ end is labeled and the 3′ end is not labeled, the OH group on the C atom at the 3′-position of the 3′ end ribose or deoxyribose may be modified with a phosphate group or the like although no limitation is imposed in this respect.

Spectroscopic, photochemical, biochemical, immunochemical, electrical, optical or chemical means can be used to detect such labels. The detection device and method may include, but is not limited to, optical imaging, electronic imaging, imaging with a CCD camera, integrated optical imaging, and mass spectrometry. Further, the amount of labeled or unlabeled probe bound to the target may be quantified. Such quantification may include statistical analysis. In other embodiments the detection may be via conductivity differences between concordant and discordant sites, by quenching, by fluorescence perturbation analysis, or by electron transport between donor and acceptor molecules.

In yet another embodiment, detection may be via energy transfer between molecules in the hybridization complexes in PCR or hybridization reactions, such as by fluorescence energy transfer (FET) or fluorescence resonance energy transfer (FRET). In FET and FRET methods, one or more nucleic acid probes are labeled with fluorescent molecules, one of which is able to act as an energy donor and the other of which is an energy acceptor molecule. These are sometimes known as a reporter molecule and a quencher molecule respectively. The donor molecule is excited with a specific wavelength of light for which it will normally exhibit a fluorescence emission wavelength. The acceptor molecule is also excited at this wavelength such that it can accept the emission energy of the donor molecule by a variety of distance-dependent energy transfer mechanisms. Generally the acceptor molecule accepts the emission energy of the donor molecule when they are in close proximity (e.g., on the same, or a neighboring molecule). FET and FRET techniques are well known in the art, and can be readily used to detect the polymorphisms of the present disclosure. See for example U.S. Pat. Nos. 5,668,648, 5,707,804, 5,728,528, 5,853,992, and 5,869,255 (for a description of FRET dyes), Tyagi et al., (1996) Nature Biotech. 14:303-8, and Tyagi et al., (1998) Nature Biotech. 16:49-53 (for a description of molecular beacons for FET), and Mergny et al. (1994) Nucleic Acid Res. 22:920-928, and Wolf et al. (1988) Proc. Natl. Acad. Sci. USA 85:8790-8794 (for general descriptions and methods fir FET and FRET), each of which is hereby incorporated by reference.

### Compositions and Kits for Detection of the SNPs of the Disclosure

The oligonucleotide primers and probes of the present disclosure have commercial applications in diagnostic kits for the detection of the ΔTTU50 polymorphism in human subjects. A test kit according to the disclosure may comprise any of the oligonucleotide primers or probes according to the disclosure. Such a test kit may additionally comprise one or more reagents for use in cyclic polymerase mediated amplification reactions, such as DNA polymerases, nucleotides (dNTPs), buffers, and the like. A ΔTTU50 polymorphism-specific detection kit may also include a lysing buffer for lysing cells contained in the specimen.

A test kit according to the disclosure, for example, may comprise a pair of oligonucleotide primers according to the disclosure and a probe comprising an oligonucleotide according to the disclosure. In some embodiments such a kit will contain two allele specific oligonucleotide probes. Advantageously, the kit may further comprise additional means, such as reagents, for detecting or measuring the binding or the primers and probes of the present disclosure, and also ideally a positive and negative control.

The present disclosure further encompasses probes according to the present disclosure that are immobilized on a solid or flexible support, such as paper, nylon or other type of membrane, filter, chip, glass slide, microchips, microbeads, or any other such matrix, all of which are within the scope of this disclosure. The probe of this form is now called a “DNA chip”. These DNA chips can be used for analyzing the ΔTTU50 polymorphism of the present disclosure. The present disclosure further encompasses arrays or microarrays of nucleic acid molecules that are based on one or more of the sequences described herein. As used herein “arrays” or “microarrays” refers to an array of distinct polynucleotides or oligonucleotides synthesized on a solid or flexible support, such as paper, nylon or other type of membrane, filter, chip, glass slide, or any other suitable solid support. In one embodiment, the microarray is prepared and used according to the methods and devices described in U.S. Pat. Nos. 5,446,603; 5,545,531; 5,807,522; 5,837,832; 5,874,219; 6,114,122; 6,238,910; 6,365,418; 6,410,229; 6,420,114; 6,432,696; 6,475,808 and 6,489,159 and PCT Publication No. WO 01/45843 A2, the disclosures of which are incorporated by reference in their entireties.

Deletion Mapping and Expression Evaluation of Genes from the Minimal Region of Deletion

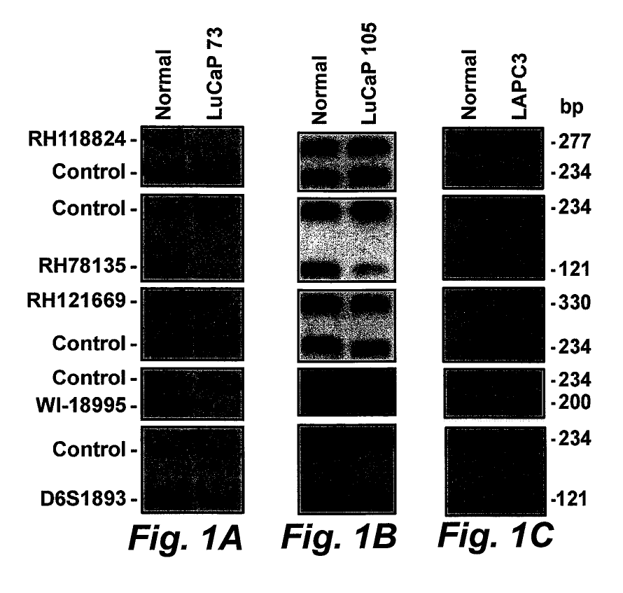

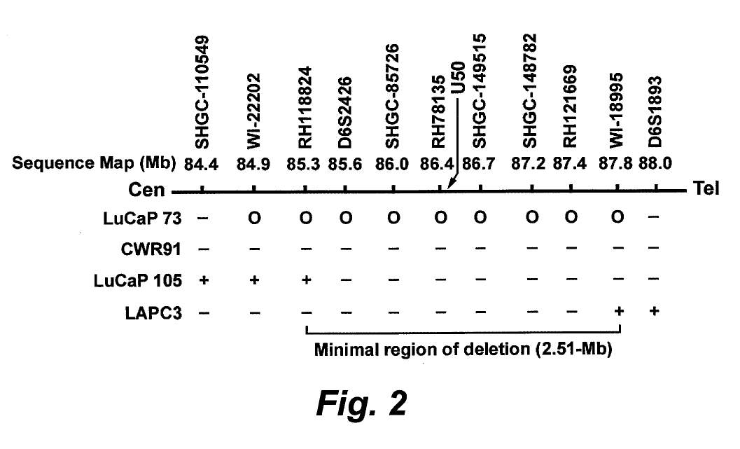

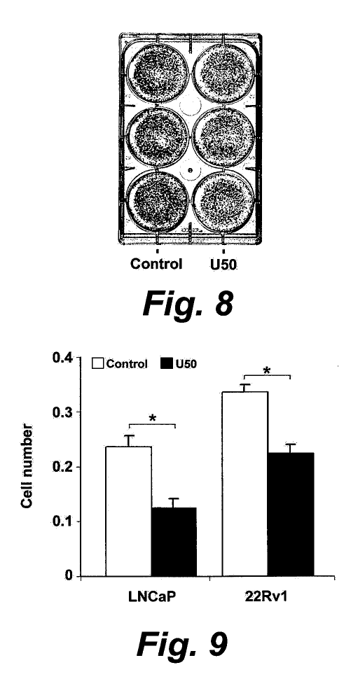

A series of assays were performed to identify the best candidate for the 6q tumor-suppressor gene. Using 30 cultured prostate cancer samples grown in culture or in mice, we were able to localize the gene to a 2.5 Mb region at 6q14-15 (FIGS. 1 and 2). Second, the expression of all the genes located in the minimal region of deletion in normal prostates were examined and excluded all but four genes for further consideration (FIGS. 4-6). Third, 30 prostate cancer samples were analyzed for cancer-specific mutations (FIGS. 13-17) and identified the snoRNA U50 as the best candidate for the 6q tumor suppressor gene because a homozygous 2 bp deletion was detected in multiple samples. Functional analysis showed that wild-type, but not mutant U50, inhibited cell proliferation or survival in the colony formation assay (shown in FIGS. 8-12). snoRNA U50 became the best candidate because it had mutations, was down-regulated and reduced colony numbers in prostate cancer.

To identify the 6q14-q22 tumor-suppressor gene(s), deletion mapping to narrow the most critical region of deletion was performed, following the approach described in Sun et al. (2005) Nat. Genet. 37:407-412, incorporated herein by reference in its entirety. Using 69 sequence-tagged site (STS) markers spanning 6q14-q22 (54.5 Mb), 30 cell lines and xenografts derived from different prostate cancers were examined to detect homozygous and hemizygous deletions by regular and duplex PCR.

A homozygous deletion of 3.6 Mb in 6q14-q15 was detected in the LuCaP 73 xenograft as shown in FIGS. 1A and 2). Hemizygous deletions overlapping with the homozygous deletion were detected in 14 of the 30 (47%) independent prostate cancers (LNCaP, PC-3, CWR21, CWR91, LAPC3, LAPC9, LuCaP 23.1/23.8/23.12, LuCaP 35/35V, LuCaP 41, LuCaP 69, LuCaP 70/70S8, LuCaP 96, LuCaP105 and LuCaP115). Although most hemizygous deletions were more extensive than the homozygous deletion, xenografts LuCaP 105 and LAPC3 had hemizygous deletions that narrowed the 3.6 Mb deletion region to 2.5 Mb at 6q14-15, between markers RH118824 and WI-18995, as shown, for example in FIGS. 1B, 1C, and 2.

Eleven verified or predicted genes lay within the 2.5 Mb 6q14-15 minimal deletion region: nine protein-coding genes (LOC389415, LOC441163, LOC441164, LOC441165, HTR1E, NT5E, SNX14, SYNCRIP and TBX18), one pseudogene (LOC401269) and one gene (U50HG) that hosts two short nucleolar RNAs (snoRNAs) named U50 and U50′. To determine which of these genes would be the best candidate(s) for the 6q14-15 tumor-suppressor gene(s), their expression in a pool of normal prostates was examined, along with 13 other normal tissues (spleen, kidney, stomach, pancreas, uterus, ovary, testis, placenta, thymus, lung, skin, adrenal gland and bone marrow) as positive controls, using the sensitive reverse-transcription PCR (RT-PCR) assay.

### Mutation Detection, Expression Evaluation and Functional Assay in Cancer Cells

For the three protein-encoding genes (LOC441164, NT5E and SYNCRIP), together with snoRNAs, U50 and U50′ that are hosted in the U50HG gene, three tests were used to determine which was most likely to be the 6q14-15 tumor suppressor gene. First, it was determined by direct DNA sequencing whether there were any mutations in any of the genes isolated from 15 prostate cancer cell lines and xenografts. Second, their expression in a panel of cell lines, xenografts and primary tumors from prostate cancer was evaluated. Third, colony functional formation assays were performed to analyze whether any of the genes could alter cell proliferation or survival.

In the 15 prostate cancer cell lines and xenografts examined, no mutations were detected for the three protein-encoding genes and the snoRNA U50′. The snoRNA U50, however, showed a homozygous two-base (TT) deletion in a stretch of four thymidines in prostate cancer xenograft LuCaP 96, as shown by a comparison of the nucleotide sequences of their respective U50-encoding genes (SEQ ID NO: 1 versus SEQ ID No: 2, respectively).

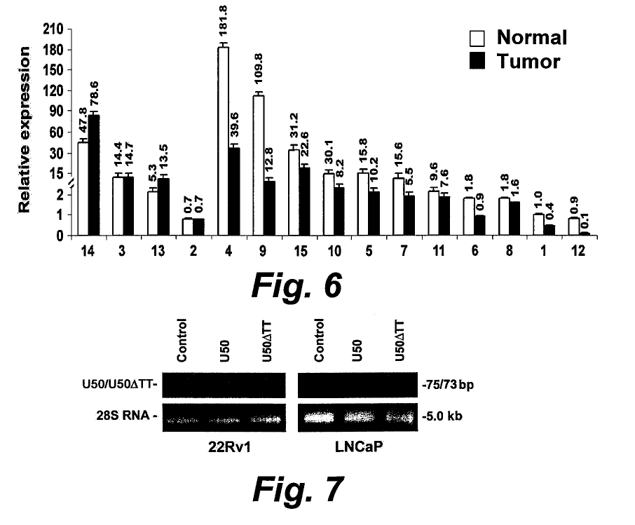

Real-time PCR analysis was used to evaluate gene expression in prostate cancer cell lines or xenografts. Compared with normal prostates, the expression of LOC441164, SYNCRIP and snoRNA U50′ was not consistently reduced in cancerous cells, although one or more cell lines showed lower levels of expression for each of them. For snoRNA U50, expression was almost completely absent in the commonly used prostate cancer cell lines 22Rv1, LNCaP and PC-3, as detected by northern blot analysis (FIG. 4). The expression of NT5E and snoRNA U50 was down-regulated in most of the prostate cancer cell lines and xenografts tested as shown, for example in FIG. 5. Human U50 expressions using real-time PCR analysis in 15 primary prostate cancer specimens were also analyzed. Compared with matched normal cells, U50 was down-regulated in 11 of the 15 cancer specimens, and the down-regulation was at least 50% in seven of them (FIG. 6).

In colony formation assays, each gene was transfected into a prostate cancer cell line in which reduced levels of expression had been demonstrated: LOC441164, NT5E, U50 and U50′ in the LNCaP cell line, and SYNCRIP in the 22Rv1 cell line. The expression of NT5E and U50 was also low in 22Rv1, so this cell line was also used to confirm the findings from LNCaP cells. Each gene was ectopically expressed with empty plasmid as the negative control. Four of the five genes, LOC441164, NT5E, SYNCRIP and snoRNA U50′, did not affect colony formation efficiency. Ectopic expression of the three protein-encoding genes in transfected cells was verified by western blot analysis using an antibody against FLAG, which was attached to the protein.

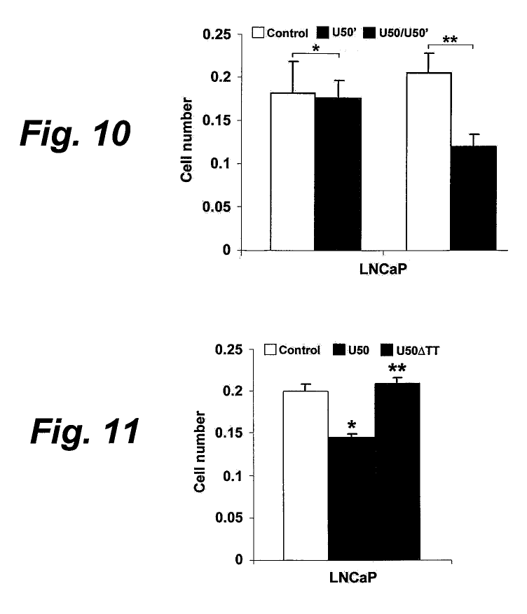

The expression of snoRNA U50 in transfected 22Rv1 and LNCaP cells was verified by northern blot assay as shown in FIG. 7. SnoRNA U50 significantly reduced colony formation in both 22Rv1 and LNCaP cell lines upon ectopic expression as shown in FIGS. 8 and 9. U50′ did not alter colony formation efficiency, whereas the combination of U50 and U50′ still significantly reduced colony formation, as illustrated in FIG. 10.

### U50 is Frequently Deleted in Breast Cancer

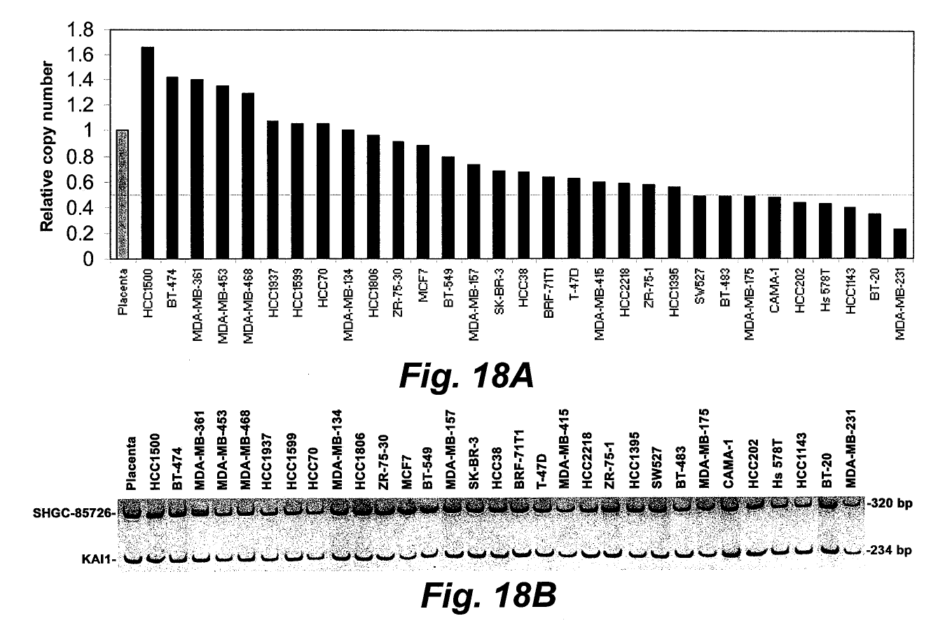

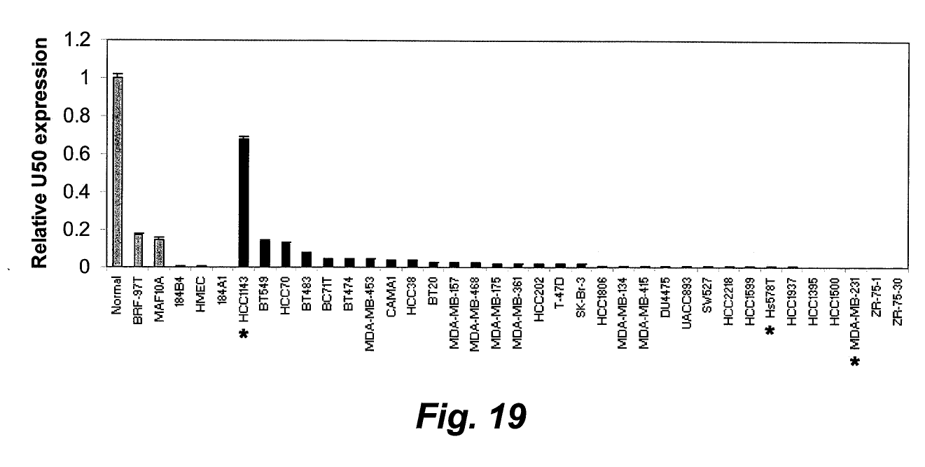

To evaluate the candidacy of snoRNA U50 as the 6q tumor suppressor gene in breast cancer, real time PCR to determine deletion frequencies of U50 in 31 breast cancer cell lines was performed. No homozygous deletions were detected. However, 9 of 31 breast cancer cell lines showed signal intensities that were less than half of that in the normal control, indicating the presence of hemizygous deletions, as shown in FIG. 18A. Duplex PCR with radioactive ³³P-dCTP was used to detect the deletion frequency of the U50 locus in these same breast cancer cell lines. Eight of the nine cell lines showing a hemizygous deletion by real time PCR assay also showed hemizygous deletion by this duplex PCR, as shown in FIG. 18B, including the cell lines BT-483, MDA-MB-175, CAMA-1, HCC202, Hs 578T, HCC1143, BT20 and MDA-MB-231. The deletion frequency of the U50 locus (25.8%) in the 31 breast cancer cell lines was similar to that reported in previous studies (Noviello et al., (1996) Clin. Cancer Res. 2:1601-1606; Schwendel et al., (1998) Br. J. Cancer 78:806-811; Seute et al., (2001) Int. J. Cancer 93:80-804), supporting the candidacy of U50 for 6q tumor suppressor gene in breast cancer.

Transcriptional Down-Regulation of snoRNA U50 in Breast Cancer

The expression of snoRNA U50 was determined in breast cancer cell lines by real time PCR analysis, with normal breast tissues and immortalized non-neoplastic mammary epithelial cell lines as controls. The highest level of U50 expression was detected in normal breast tissues. However, reduced U50 expression was detected in the four immortalized non-neoplastic mammary epithelial cell lines, and in all the breast cancer cell lines tested except for HCC1143 (FIG. 19). Compared to normal breasts, the reduction of U50 expression in all the 37 cell lines tested (except for HCC1143) was by at least 80%, and some cell lines had no detectable expression at all. In the HCC1143 cell line, which is the only breast cancer cell line expressing a high level of U50, mutation analysis revealed that U50 was homozygously mutated. All the breast cancer cell lines with a hemizygous deletion (except for HCC1143) showed a reduction of expression by more than 96%.

### Detection of U50 Mutation in Prostate Cancer Samples

On the basis of the results of mutation, expression loss and functional effect on cell proliferation or survival, as shown in FIGS. 7-12, U50 was the primary candidate for the 6q14-15 prostate cancer tumor suppressor. To further evaluate the candidacy of U50, PCR combined with a single-strand conformation polymorphism (SSCP) assay, direct DNA sequencing and denaturing polyacrylamide gel electrophoresis to detect mutations in the 30 prostate cancer cell lines and xenografts available was used. In addition to LuCaP 96, the same homozygous TT deletion was also detected in xenograft LAPC3 (FIG. 13). Meanwhile, a heterozygous TT deletion was detected in cell line NCI-H660 and xenograft LuCaP 86.2 (FIG. 13). In addition, a one-base deletion in a stretch of 11 adenines in the neighborhood of the four thymidines in the U50 genome sequence SEQ ID NO: 1 (FIG. 3A) was detected in two other xenografts, LAPC4 and LuCaP 58. Although it is not clear whether this one-base deletion in the polyA tract affects U50 function, it is likely that it results from a defective mismatch repair system on the basis of our previous findings that both LAPC4 and LuCaP 58 had microsatellite instability and none of the 89 localized prostate cancers and 104 control samples had the deletion of the polyA tract. Neither did the 1371 men with prostate cancer and 1371 matched control men have this one-base deletion, on the basis of the genotyping results. None of the four samples with a TT deletion had any change in the polyA tract, consistent with the fact that none of them had microsatellite instability.

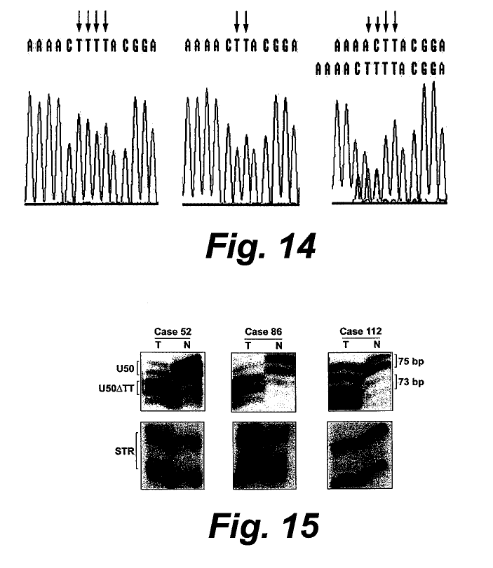

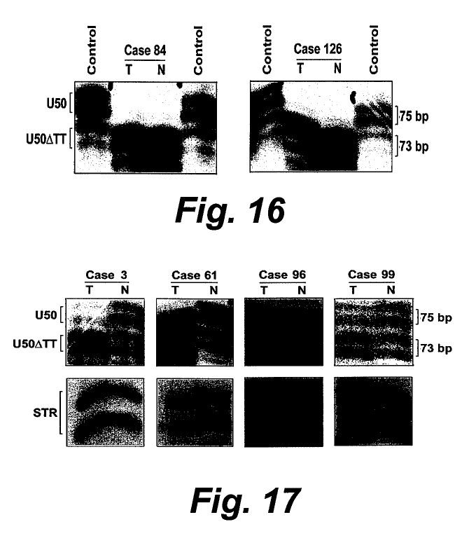

To further evaluate the role of U50 in prostate cancer, 89 grossly dissected primary prostate cancers, with matched non-cancer cells as controls were examined. In total, nine of 89 (10%) tumors showed a homozygous genotype for the TT deletion in tumor cells (FIGS. 14-17). These nine deletions appear to be a combination of more somatic alterations (7/89 or 8%) and less germline polymorphisms (2/89 or 2%). In three of the nine tumors with a homozygous TT deletion, the matched normal cells showed only the wild-type allele, which indicates that the TT deletion occurred somatically in these cases (FIG. 16). Two of the nine tumors also showed a homozygous TT deletion in their matched non-cancer cells, indicating that the mutation occurred in the germline of these men (FIG. 15).

For the remaining four of the nine tumors with homozygous TT deletion, their matched normal cells showed a heterozygous genotype for the mutation (FIG. 17), indicating that, during carcinogenesis, the wild-type allele was either mutated somatically, as in cases 52, 86 and 112, or lost through somatic deletion of 6q14.3. Loss of heterozygosity is common at 6q14-15 in prostate cancer, and, at random, both wild-type allele and the allele with the deletion should be lost at an equal frequency. The fact that the loss or somatic mutation only occurred in the wild-type allele but not in the mutant allele in the cases with a germline heterozygous genotype further suggests that loss of the wild-type U50 allele provides a survival advantage for cancer cells.

In addition to the nine cases with homozygous TT deletion in tumor cells, five of the 89 (6%) cases that showed a heterozygous genotype in both normal and cancer cells, which brought the total number of cases with a heterozygous genotype to nine (10% of the 89 samples), further indicating that the TT deletion can be a germline event. To ensure that DNA samples for normal and cancer cells in the seven cases showing somatic mutation or deletion of the wild-type U50 allele in cancer cells were from the same individual, each pair was analyzed using the AmpFLSTRw Identifilerw PCR Amplification Kit that has been optimized for human identification. Each pair of normal and cancer cells were from the same individual (FIGS. 16 and 17), excluding mismatching the samples for the TT deletion, and indicating that homozygous TT deletion in U50 is a cancer-related alteration. No association was found between U50 homozygous deletion and clinicopathological characteristics of the clinical samples analyzed, including tumor grade, tumor stage and recurrence.

In total, 11 of 119 (9%) prostate cancers examined had the homozygous TT deletion, as shown in Table 1, Example 7. Seven of the 11 cases involved a somatic alteration, two resulted from a germline mutation and the two in cell lines/xenografts had an unknown origin because no DNA from matched normal cells was available for analysis. Considering that the mutation also occurred in germline, we evaluated the incidence of TT deletion in a normal population. 104 control men who did not have prostate cancer at the time of blood collection were examined. None of the 104 (0%) control men showed a homozygous TT deletion in U50. Because the prostates from the 104 control men did not have detectable cancer and deletion of 6q is primarily somatic in prostate cancer, we could thus compare the 89 cases and 104 controls to determine cancer-specific incidence of homozygous U50 deletion in sporadic prostate cancer. The frequency of 7/89 or 8% for the homozygous U50 deletion was significantly higher than the 0/104 incidence in the controls (P=0.02, χ² test), indicating that somatic U50 mutation is indeed a cancer-specific alteration.

On the other hand, 12 of the 104 control samples (12%) showed the presence of both a wild-type allele and the TT deletion allele, which is similar to the incidence of nine of 89 (10%) in human subjects with prostate cancer described earlier and thus raises the possibility that the TT deletion could be a benign polymorphism. To further evaluate whether the TT deletion is a cancer-related mutation or a benign polymorphism, U50 expression and performed functional assays were conducted. Knock-down of the expression of U50 (75 bases) by RNA interference in the DU-145 and RWPE1 prostatic cell lines, which express higher levels of U50, was conducted.

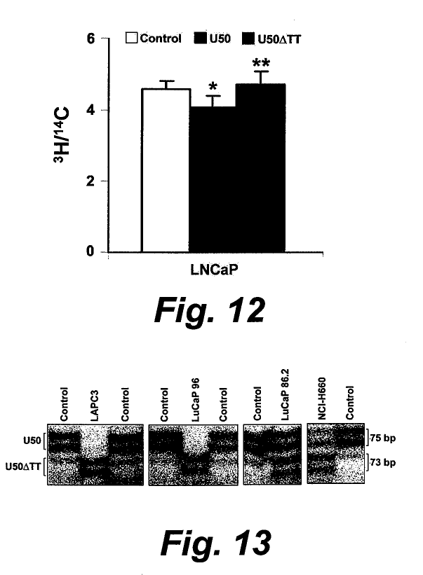

Two small interfering RNAs: 5′-CCUGAACUUCUGUUGAA AA-3′ (SEQ ID NO: 5) and 5′-ACUUUUACGGAUCUGGCUU-3′ (SEQ ID NO: 6) were tested but did not alter U50 expression or caused any changes in colony formation. Wild-type and mutant U50, along with the vector control, in LNCaP prostate cancer cells were also expressed, and colony formation and cell proliferation assays were performed. As illustrated in FIGS. 11 and 12, the TT deletion abolished the function of U50 in suppressing colony formation and cell proliferation, indicating that the mutation affects its function. The expression of U50 and its mutant in transfected cells was verified by northern blot analysis (FIG. 7).

Mutations of snoRNA U50 in Breast Cancer

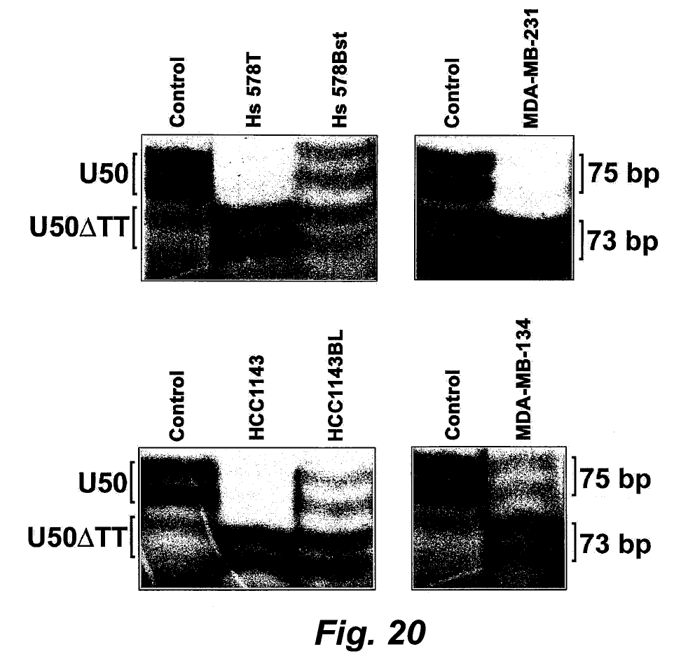

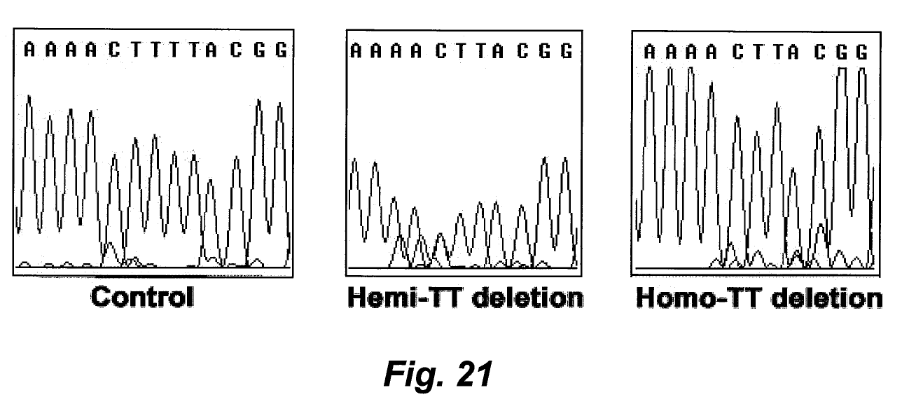

The nature of U50 mutations in 31 breast cancer cell lines were first analyzed by PCR combined with denaturing polyacrylamide gel electrophoresis and direct sequencing. U50 showed a homozygous 2-base (TT) deletion in the stretch of 4 thymidines in three breast cancer cell lines (9.7%, in the cell lines HCC1143, Hs 578T and MDA-MB-231) and hemizygous TT-deletions in one breast cancer cell line (MDA-MB-134), as shown in FIG. 20. Because the TT-deletion occurred in germline tissues in prostate cancer samples the mutations in breast cancer cell lines were also evaluated as to whether they were germline or somatic.

The U50 genotypes were determined for cell lines established from peripheral blood cells from four of the breast cancer cell lines (HCC38BL, HCC1143BL, HCC1937BL and Hs 578Bst) that were obtained from the same women from whom the breast cancer cell lines HCC38, HCC1143, HCC1937 and Hs 578T were derived. Compared to HCC1143 and Hs 578T, which showed homozygous deletion of U50, their matched blood cells HCC1143BL and Hs 578Bst showed a hemizygous deletion (FIG. 20), suggesting that the wild-type allele of U50 in these two subjects was either lost through loss of heterozygosity (LOH) or mutated during the development of their breast cancers. Consistent with this result, both HCC1143 and Hs 578T has hemizygous deletion at U50 (FIG. 18).

Two other lymphocyte lines, HCC38BL and HCC1937BL, showed a wild-type U50, same as their matched breast cancer cell lines HCC38 and HCC1937. For the MDA-MB-231 breast cancer cell line, which is also homozygous for the deletion, the origin of the mutation could not be determined due to lack of matched normal genomic DNA. However, LOH could also have given rise to the mutation because it had a hemizygous deletion at U50 (FIG. 18).

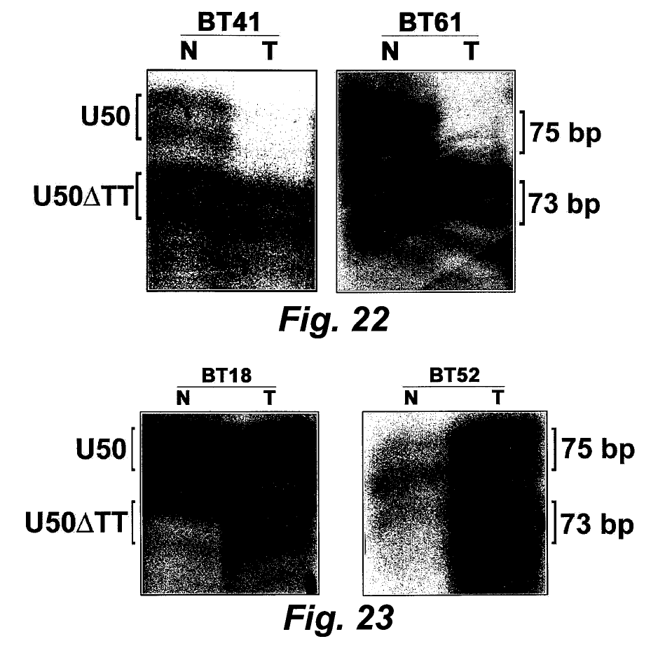

U50 mutations in cancer cells and matched non-cancer cells from 49 primary breast cancer samples were then examined. Two of the 49 (4.1%) cases showed a homozygous genotype of the TT-deletion in their tumor cells, but they were hemizygous genotype in their matched normal cells (FIG. 22), indicating that the TT-deletion occurred somatically in these cases. Another 2 of the 49 cases (4.1%) showed a hemizygous genotype for the TT-deletion in their tumor cells (shown in FIG. 23), while their matched normal cells showed a wild-type genotype, indicating that one of the two U50 alleles was mutated in these tumor.

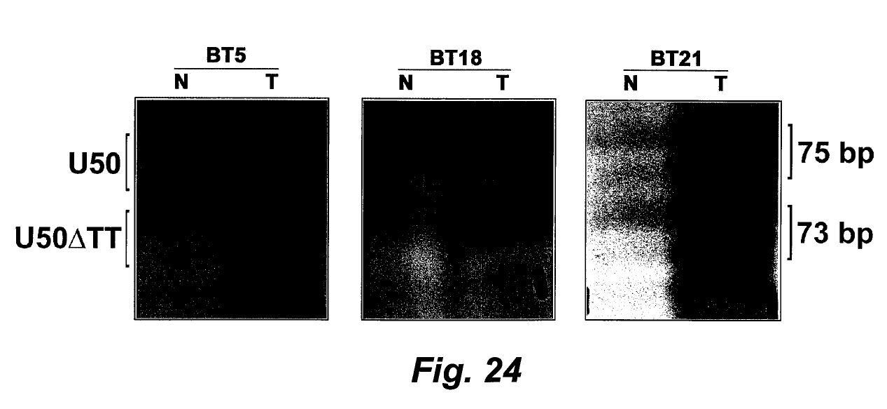

In 3 of the 49 cases, both cancer cells and matched non-cancer cells showed a hemizygous genotype for the TT-deletion (FIG. 24), further indicating that the TT-deletion in U50 occurs in germline cells. None of the 49 samples had wild-type U50 in tumor cells but a deletion in normal cells.

Loss of heterozygosity is common at 6q14.3-15 in breast cancer and, at random, both wild-type allele and the allele with deletion should be lost at an equal frequency. The fact that the loss or somatic mutation only occurred in the wild-type allele but not in the mutant allele in the cases with a germline heterozygous genotype suggests that loss of the wild-type U50 allele provides a survival advantage for breast cancer cells.

### Association Study of the U50 Mutation in a Cohort of Cases and Controls

To further determine the role of the U50 deletion in prostate cancer and rule out the possibility of homozygous TT deletion as a benign polymorphism, 1371 men with prostate cancer and 1371 matched control men for the 2 bp TT deletion were genotyped and associated different genotypes with prostate cancer and clinically significant prostate cancer, using a well established epidemiologic cohort reported previously (Calle et al., (2002) Cancer 94:2490-2501; Patel et al., (2005) Breast Cancer Res., 7:R1168-R1173). Both prostate cancer cases and controls in this analysis were predominantly white (approx. 99% of both cases and controls) and elderly at the time of diagnosis (median age 70 years). Genotype distribution and results of regression models are presented in Table 2, Example 8. In the analysis adjusted for the matching factors, men homozygous for the 2 bp deletion had an increased risk of being diagnosed with prostate cancer that was not statistically significant [odds ratio (OR) 1.85, 95% confidence interval (CI) 0.85-4.03].

Clinically significant prostate cancers were separated from total prostate cancers. Clinically significant prostate cancer was defined by Gleason score≧7 or grade 3-4, stage C or D at diagnosis, or men who had prostate cancer as their underlying cause of death. The risk of clinically significant prostate cancer was significantly increased among men who were homozygous for the deletion (OR 2.63, 95% CI 1.08-6.38) (as shown in Example 8, Table 2). Having a single copy of the deletion (heterozygous) was not significantly associated with risk of total or clinically significant prostate cancer. Results did not change meaningfully when we adjusted for prostate cancer risk factors in this study population.

Some of the control men, although cancer-free at the time of the diagnosis of their matched cases, were diagnosed with prostate cancer during subsequent follow-ups. When these men (n=24) were excluded in statistical analysis, the association between homozygous deletion and risk of total prostate cancer (OR 2.03; 95% CI 0.91-4.55) and clinically significant prostate cancer (OR 2.90; 95% CI 1.17-7.21) was stronger.

### Homozygous Deletion of U50 Occurs Both Somatically and in Germline

U50 was examined in 89 localized prostate cancers for its role in sporadic prostate cancer and for better evaluation of its candidacy for the 6q 14-15 tumor-suppressor gene. Seven of the 89 (8%) cancers showed the same homozygous deletion, whereas none of their matched normal cells did. Among the seven cancer-specific homozygous deletions, three originated from mutation and four originated from either mutation or chromosomal loss (FIGS. 13-17). Chromosomal loss is common at 6q14.3-15 in prostate cancer, but the loss at U50 did not occur in any of the nine prostates that were heterozygous for the deletion allele, suggesting that homozygosity of the deletion at U50 is selected during carcinogenesis.

The results show that the homozygous deletion of U50 plays a role in prostatic carcinogenesis. In addition to the seven localized prostate cancers with somatic alterations in U50, two of the 89 cancers had the homozygous deletion in both their normal and cancer cells, which brought the total number of cancers with the homozygous deletion to nine (10% of the 89 cases). None of 104 verified cancer-free men had the homozygous deletion in their germline DNA. More frequent homozygous deletion of U50 in cancers further supports U50 for the 6q14-15 tumor-suppressor gene. These results show that homozygous deletion of U50 is involved in approximately 10% of sporadic prostate cancers.

### U50 Could be a Typical Recessive Tumor-Suppressor Gene

In inactivation of a recessive tumor-suppressor gene, both alleles need to be mutated and/or deleted, which is referred to as ‘two hits’, to functionally inactivate a tumor-suppressor gene. The first hit is often a germline mutation, whereas the second hit is a somatic mutation or allelic loss. The results in this study suggested that U50 is a typical recessive tumor-suppressor gene that requires the loss of both wild-type alleles, or ‘two hits’, to be inactivated in cancer. The relatively common germline TT deletion in one of the two alleles, as seen in approximately 10% of the populations that had a heterozygous genotype for the TT deletion, could be the first hit. The first hit may be recessive and has no effect on U50 function when the wild-type allele is present. When the second hit occurs through either somatic mutation or chromosomal deletion or germline mutation in some cases, as described in this study, U50 can be inactivated and contribute to the development of prostate cancer, because a homozygous but not heterozygous genotype of the deletion was significantly associated with clinically significant prostate cancer.

As indicated, for example, by the data presented in Tables 1 and 2, below, for prostate cancer and described for breast cancer, the 2 bp homozygous deletion also occurs in germline, i.e., some individuals inherit this deletion from their parents. In prostate cancer, inheritance of one mutant allele does not appear to increase the risk of prostate cancer, but when both maternal and paternal alleles have the U50 deletion (i.e. homozygous deletion of the 2 bp in U50), the risk of prostate cancer is significantly increased. In breast cancer, inheritance of even one allele with a U50 deletion increases the risk of breast cancer. Therefore, inheritance of two alleles in men, or one allele in women, increases the risk of prostate cancer and breast cancer.

Based on these findings, the present disclosure provides a method, suitable for use in a genetic clinical setting, to analyze the allele status of U50 in blood DNA from individuals who do not have cancer. If a male subject has two mutant U50 alleles, or a female has just one mutant allele, he or she is indicated as being at increased risk of developing prostate cancer or breast cancer, respectively. Preventive intervention may be prescribed to lower such risk.

Malfunction of snoRNA and Oncogenesis

Small nucleolar RNAs represent a common class of non-coding RNAs abundantly expressed in mammalian cells. They constitute a major component of small nucleolar ribonucleoprotein complexes and guide site-specific modifications of nucleotides in target RNAs (Kiss. T., (2002) Cell 109:145-148). The U50 snoRNA is one of over 300 known human snoRNAs. It is encoded by intron 5 of the U50HG gene. An snoRNA gene can be located at a chromosomal breakpoint involved in carcinogenesis. For example, the U50 snoRNA was originally discovered from the breakpoint of chromosomal translocation t (3,6) (q27;q15), which is involved in human B-cell lymphoma. A recent study demonstrated that adeno-associated viruses integrate their genome into mouse genome, which causes liver cancer, and the integration sites identified in tumors were all located within a DNA interval encoding some snoRNAs.

The expression of snoRNA has been associated with growth arrest of prostate and breast cancer cells. For example, the host gene for U50, U50HG, possesses an oligopyrimidine tract that is characteristic of the 50-terminal oligopyrimidine (50TOP) class of genes, which have been shown to be coordinately regulated in response to cell growth. The gas5 gene, which hosts multiple snoRNAs, is also a member of the 50TOP gene family and has been reported as a growth arrest-specific gene, because the accumulation of gas5-generated snoRNAs was associated with an arrest of cell growth, consistent with the results in this study and indicate that snoRNA could be associated with growth arrest and likely tumor suppression. A common region of deletion in 6q14-15 was identified, all expressed genes in the common region for cancer-specific mutations were evaluated and the snoRNA U50 having a homozygously 2 bp deletion in approximately 10% of sporadic prostate cancers was identified. Furthermore, homozygous genotype of the deletion was significantly associated with clinically significant prostate cancer in a prospectively analyzed cohort of prostate cancer cases and controls. The findings, therefore, indicate that snoRNA U50 is a reasonable candidate for the 6q14-15 tumor suppressor gene in human prostate and breast cancer, its homozygous deletion is involved in approximately 10% of sporadic prostate cancers and that germline homozygosity of the deletion could predict clinically significant prostate cancer.

The present disclosure encompasses methods of diagnosing the presence of a cancer of the prostate or breast in a human subject, predicting the likelihood of developing a prostate or breast cancer, predicting the outcome or severity of the disease and methods of reversing the prostate cell transformation based on the presence or absence in the human subject of a dinucleotide (TT) deletion in the gene encoding the U50 snoRNA.

One aspect of the present disclosure, therefore, provides methods of identifying a genetic marker of a human subject indicating a cancerous tissue in the human subject, embodiments of the methods comprising: obtaining an isolated nucleic acid sample from a human subject; and determining from the isolated nucleic acid sample the genotype of the human subject with respect to a locus encoding a snoRNA U50, whereby a mutation within the nucleotide sequence encoding a snoRNA U50, when compared with a wild-type nucleotide sequence encoding a snoRNA U50, identifies in the human subject a genetic marker associated with a cancer in the human subject.

In embodiments of this aspect of the disclosure, the nucleotide sequence encoding a snoRNA U50 may comprise the nucleotide sequence according to SEQ ID NO: 1.

In embodiments of the disclosure, the wild-type U50 nucleic acid sequence may comprise the nucleotides 47-60 of the nucleotide sequence according to SEQ ID NO: 1.

In embodiments of the methods of this aspect of the disclosure, the mutation can be a TT dinucleotide deletion from within a nucleotide region comprising nucleotide position 47 to position 60 of the nucleotide sequence according to SEQ ID NO: 1, and wherein the mutation is associated with a cancer.

In the embodiments of the disclosure, the cancer may be a prostate cancer or a breast cancer.

In the various embodiments of the disclosure, the step of determining from the isolated nucleic acid the genotype of the biological sample with respect to a U50 locus encoding a snoRNA U50 may comprise: isolating by PCR amplification a nucleic acid molecule comprising the nucleotide sequence from nucleotide position 47 to position 60 of the nucleotide sequence according to SEQ ID NO: 1; and determining whether the nucleic acid molecule has a dinucleotide deletion within the nucleotide sequence from nucleotide position 47 to position 60 of the nucleotide sequence according to SEQ ID NO: 1 when compared to a wild-type control nucleotide sequence.

In embodiments of this method of the disclosure, the PCR amplification may use oligonucleotide primers having the nucleotide sequences according to SEQ ID NOs: 3 and 4.

In one embodiment of the disclosure, determining whether the nucleic acid molecule has dinucleotide deletion within the nucleotide sequence from nucleotide position 47 to position 60 of the nucleotide sequence according to SEQ ID NO: 1 when compared to a wild-type control nucleotide sequence, is by a single-base extension reaction.

In this embodiment, the single-base extension reaction may use a primer having a nucleotide sequence selected from the group consisting of SEQ ID NOs: 3 and 4.

In various embodiments of this aspect of the disclosure, the isolated nucleic acid from the human subject and a first oligonucleotide probe having a nucleotide sequence capable of specifically detecting a mutation within a nucleotide sequence of the isolated nucleic acid encoding an snoRNA U50 are hybridized under conditions allowing the first probe to specifically hybridize to the isolated nucleic acid sample if the nucleotide sequence encoding the snoRNA U50 has a mutation therein with a cancer.

In embodiments of the disclosure, the first oligonucleotide probe may comprise the nucleotide sequence according to SEQ ID NO: 19.

In embodiments of the disclosure, the first oligonucleotide is capable of specifically hybridizing under stringent conditions to a nucleic acid molecule comprising the nucleotide sequence according to SEQ ID NO: 2.

In embodiments of the disclosure, the methods may further comprise hybridizing the isolated nucleic sample with a second oligonucleotide probe having a nucleotide sequence capable of specifically detecting under high stringency conditions a nucleotide sequence encoding an snoRNA U50, wherein the nucleotide sequence encoding the snoRNA U50 does not have a mutation therein with a cancer. In these embodiments, the second oligonucleotide comprises the nucleotide sequence according to SEQ ID NO: 18.

In embodiments of this aspect of the disclosure, the methods may further comprise correlating the presence of the genetic marker in the gene locus encoding the snoRNA U50 with the prognostic outcome for a prostate cancer in the human subject.

In other embodiments of the disclosure, the methods may further comprise correlating the presence of the genetic marker in the gene locus encoding the snoRNA U50 with the presence or absence of a breast cancer in the human subject.

In other embodiments, the methods of this aspect of the disclosure may further comprise correlating the presence of ΔTT genetic marker in the gene locus encoding the snoRNA U50 with a probability of the human subject developing a prostate or a breast cancer.

Yet another aspect of the disclosure provides a method of modifying the proliferative status of a cell by introducing into the cell a nucleic acid molecule comprising a sequence comprising the sequence of nucleotides from nucleotide position about 47 to about position 60 of the nucleotide sequence according to SEQ ID NO: 1.

In embodiments of this aspect of the disclosure, the nucleic acid molecule may comprise the nucleotide sequence according to SEQ ID NO: 1.

In other embodiments, the introduction into the cell of the nucleic acid molecule reduces the proliferation of the cell.

In other embodiments of the disclosure, the cell may be selected from the group consisting of a prostate cancer cell and a breast cancer cell.

Another aspect of the disclosure provides embodiments of a kit for determining whether a biological sample from a human subject has dinucleotide deletion within a nucleic acid region encoding the snoRNA U50, wherein the kit may comprise at least one oligonucleotide comprising a nucleotide sequence selected from the group consisting of the nucleotide sequences according to SEQ ID NOs: 3, 4, 5, 6, 7, 18 and 19, and instructions for determining whether an isolated nucleic acid sample from a human subject has cancer-associated mutation within a nucleotide region encoding snoRNA U50.

Unless otherwise defined, all technical and scientific terms used herein have the same meaning as commonly understood by one of ordinary skill in the art of molecular biology. Although methods and materials similar or equivalent to those described herein can be used in the practice or testing of the present disclosure, suitable methods and materials are described herein. All publications, patent applications, patents, and other references mentioned herein are incorporated by reference in their entirety. In addition, the materials, methods, and examples are illustrative only and are not intended to be limiting.

The following examples are provided to describe and illustrate, but not limit, the claimed disclosure. Those of skill in the art will readily recognize a variety of non-critical parameters that could be changed or modified to yield essentially similar results.

### EXAMPLES

### Example 1

### Cell Lines, Xenografts, Tissue Specimens and Blood DNA Samples

Six prostate cancer cell lines (DU-145, NCI-H660, LNCaP, 22Rv1, MDAPCa2b and PC-3) and two immortalized and untransformed prostatic epithelial cell lines (PZ-HPV7 and RWPE1) were purchased from the American Type Culture Collection (Manassas, Va., USA). Cells were propagated following standard protocols from ATCC. Twenty-seven xenografts from 24 prostate cancers (Sun et al., (2006) Prostate 66:660-666, incorporated herein by reference in its entirety) were also used, including CWR21, CWR22, CWR91, LAPC3, LAPC4, LAPC9, PC82, LuCaP 23.1, LuCaP 23.8, LuCaP 23.12, LuCaP 35, LuCaP 35V, LuCaP 41, LuCaP 49, LuCaP 58, LuCaP 69, LuCaP 70, LuCaP 73, LuCaP 77, LuCaP 78, LuCaP 81, LuCaP 86.2, LuCaP 92.1, LuCaP 93, LuCaP 96, LuCaP 105 and LuCaP 115.

For mutation analysis, genomic DNA for matched cancer and normal cells was isolated from 89 localized prostate cancers that were treated by prostatectomy and did not have lymph node involvement or distant metastasis at the time of surgery. Briefly, 10 consecutive sections were cut from each tissue block and mounted on slides. The first one was cut at 5 mm and stained with hematoxylin to identify tumor and normal cells from each sample. Sections 2-10 were cut at 12 mm and stained with hematoxylin. Regions rich in tumor cells were microdissected from these sections, and the surrounding normal tissues were also isolated from the same slides as matched normal cell controls. DNA isolation was as described previously (Sun et al., (2006) Prostate 66:660-666, incorporated herein by reference in its entirety).

Total RNA samples from normal human prostates and 13 other normal tissues (Clontech, Palo Alto, Calif., USA) were used for expression analysis. In addition, total RNA was isolated from 15 fresh prostate cancers and used for expression analysis. Briefly, fresh prostate tissue was sectioned with a sterile scalpel blade to identify and collect a piece of cancer tissue into RNAlater solution (Ambion, Austin, Tex., USA). A piece of normal tissue was also collected. After pathological verification of the tissue, total RNA was isolated following a standard protocol. Finally, genomic DNA from the blood cells of 104 unrelated individuals without any cancer was used to evaluate U50 germline mutation. Genomic DNA for all the samples and RNA for all the cell lines and some of the xenografts were extracted following standard procedures.

### Example 2

### Prospective Study of U50 Mutation in Prostate Cancer

Men in the association analysis were participants in the Cancer Prevention Study II (CPS-II) Nutrition Cohort, a prospective study of cancer incidence including approximately 184 000 US men and women, established by the American Cancer Society (Calle et al., (2002) Cancer 94:2490-2501, incorporated herein by reference in its entirety). At enrollment into the Nutrition Cohort in 1992 or 1993, all participants completed a self-administered questionnaire that included questions on demographic, medical and life-style factors. Most participants were 50-74 years at the time of enrollment. Beginning in 1997, follow-up questionnaires were sent to cohort members every 2 years to update exposure information and to ascertain newly diagnosed cancers. Incident cancers reported on questionnaires were verified through medical records, linkage with state cancer registries or death certificates. The recruitment, characteristics, and follow-up of the CPS-II Nutrition Cohort are described in detail elsewhere (Calle et al., (2002) Cancer 94:2490-2501, incorporated herein by reference in its entirety). From June 1998 through June 2001, participants in the CPS-II Nutrition Cohort were invited to provide a blood sample. After obtaining informed consent, blood samples were collected from 39,071 participants, including 17,411 men. Among men who had provided a blood sample, we identified 1452 cases that had been diagnosed with prostate cancer between 1992 and 2003 and had not been diagnosed with any other cancer (other than non-melanoma skin cancer).

For each case, one control was selected from men who had provided a blood sample and were cancer-free at the time of the case diagnosis. Each control was individually matched to a case on birth date (+6 months), date of blood collection (+6 months) and race/ethnicity (white, African/American, Hispanic, Asian, other/unknown). A total of 81 prostate cancer cases and 81 of the controls initially selected were later excluded because of low DNA or contaminated sample. A total of 1371 cases and controls remained for analysis. Among the cases defined were clinically significant prostate cancer (534 cases) as those with Gleason score 7 or grade 3-4, stage C or D at diagnosis or men who had prostate cancer as their underlying cause of death.

### Detection of Homozygous and Hemizygous Deletions

A total of 69 STS markers spanning the region of 6q14-q22 were used to detect homozygous and hemizygous deletions by regular and duplex PCR, as described in our previous study (Sun et al., (2005) Nat Genet. 37:407-412). A hemizygous deletion was considered to be present when the ratio of signal intensity for a 6q marker to that for the control marker in a tumor sample was less than half of the ratio in the normal human placenta DNA (Clontech) or matched normal cells. The control marker was from exon 5 of the KAI1 gene, which is rarely altered at the genomic level in human prostate cancer.

### Example 3

### Expression Analysis

Total RNA was converted into cDNA using the Iscript cDNA synthesis kit (Bio-Rad Laboratories, CA, USA) according to the manufacturer's protocol. PCR amplification was then performed on the cDNA, with primers spanning different exons of different genes except for U50. The forward and reverse primer sequences, respectively, were:

| | |
|---|---|
| for LOC441164: | |
| 5′-ACTGAAGACAGCGCCATTGTTCCTG-3′ | (SEQ ID NO: 8) |
| and | |
| 5′-GGGTGGTAGGTGAGTGGGTATTGCG-3′; | (SEQ ID NO: 9) |
| for NT5E: | |
| 5′-TGGGCGGAATCCATGTGGTGTATG-3′ | (SEQ ID NO: 10) |
| and | |
| 5′-TCCACCATTGGCCAGGAAGTTTGG-3′; | (SEQ ID NO: 11) |

for SYNCRIP: 5′-TACCTCCACGCCCTCGACC-3′ (SEQ ID NO: 12 and 5′-AGCTGGACCTATATGGGATCTTCG-3′ (SEQ ID NO: 13). For the expression analysis of U50 by PCR, a primer with a linker sequence attached to a U50-specific sequence (5′-TCGAGCGGCCGCCCGGGCAGGTATCTCAGAAGCCAGATCCG-3′ (SEQ ID NO: 3; linker sequence is in boldface), along with a primer specific for GAPDH (5′-GTGGTCCAGGGGTCTTACTC-3′ (SEQ ID NO: 14)), was used to direct cDNA synthesis using the SuperScript II reverse transcriptase (Invitrogen, Carlsbad, Calif., USA).

The following pairs of primers, 5′-TCGAGCGGCCG CCCGGGC-3′ (SEQ ID NO: 15) (complementary to the linker sequence) and 5′-TATCTGTGATGATCTTATCCCGAACCTG AAC-3′ (SEQ ID NO: 16) for U50, and 5′-GTGGTCCAGGGGTCTTACTC-3′ (SEQ ID NO: 14) and 5′-TTCAACAGCGACACCCACTC-3′ (SEQ ID NO: 17) for GAPDH, were used to detect gene expression. In addition to regular RT-PCR, we also performed real-time PCR with the ABI SYBR Green Kit and the ABI Prism 7000 Sequence Detection System (Applied Biosystems, Foster City, Calif., USA) to detect gene expression in prostate cancer samples. Expression of a gene in each sample was indicated by the ratio of gene-specific reading to the reading of GAPDH, which was normalized by the normal control. In the northern blot analysis for U50 and U50DTT expression, 15 mg total RNA for each sample was separated by gel electrophoresis in a 6% denaturing polyacrylamide gel containing 7 M urea, transferred to Hybond-C nylon membrane (Amersham) and hybridized with 32P-labeled probe in QuikHyb Hybridization solution (Stratagene, La Jolla, Calif., USA) following standard protocols. The probes were generated by PCR amplification with primers used for U50 and U50DTT expression constructs and radiolabeled by PCR amplification in the presence of 32P-dCTP with the primer complementary to U50 (5′-ATCTCAGAAGCCAGATCCGTAAAAG-3′ (SEQ ID NO: 18)) or U50DTT (5′-ATCTCAGAAGCCAGATCCGTAAG-3′ (SEQ ID NO: 19)). The same amount of RNA for each sample was separated on a denaturing agarose gel for 28S RNA as a loading control.

### Example 4

### Colony Formation and Cell Proliferation Assays

The coding regions for LOC441164, NT5E and SYNCRIP were cloned into the FLAG-pcDNA3 expression vector (Invitrogen). The in-frame FLAG tag enabled the detection of protein expression by western blot analysis with anti-FLAG antibody (Sigma). On the basis of previous studies, a tag did not appear to affect the function of SYNCRIP in different analyses (Cho et al., (2007) Mol. Cell Biol. 27:368-383, incorporated herein by reference in its entirety). Therefore, the in-frame FLAG tag in our study should not affect SYNCRIP function either. For NT5E, a FLAG-tagged construct was transfected into the MDA-MB-231 breast cancer cells and performed colony formation assay. The results with a tagged NT5E were similar to that from untagged NT5E in a previous study (Zhi et al., (2007) Clin. Exp. Metastasis 24:439-448), incorporated herein by reference in its entirety), which indicates that the FLAG tag did not affect NT5E function in the study. For LOC441164, it is not clear whether a FLAG tag affects its function or not. U50, its mutant with the TT deletion (U50DTT) and U50′ sequences were cloned into the pSIRENRetroQ vector (Clontech), which was designed to accurately express small RNA molecules.

For U50, the 22Rv1 and LNCaP prostate cancer cell lines, which express little U50, were seeded into six-well tissue culture plates. The next day, the Lipofectamine Plus reagent (Invitrogen) was used to transfect 1.6 mg of pSIRENRetroQ-U50 plasmid or the pSIREN-RetroQ vector control into cells. Forty-eight hours after transfection, puromycin was added into the media at a final concentration of 2 mg/ml, which completely killed parental 22Rv1 or LNCaP cells in 12 days. One set of cells were used to verify the expression of U50 by real-time PCR and northern blot analysis. At days 8 and 12 after selection started, cells were fixed and stained with sulforhodamine B, and optical densities, which indicated cell numbers, were measured as described previously (Sun et al., (2006) Prostate 66:660-666, incorporated herein by reference in its entirety). U50′ and U50DTT were analyzed in the same manner. The effect of U50 or U50DTT on the proliferation of LNCaP cells was determined by measuring ³H-thymidine incorporation following a standard protocol. Briefly, LNCaP cells were seeded in 24-well plates with the medium containing ¹⁴C-thymidine. On the following day, cells were washed three times with PBS to remove free ¹⁴C-thymidine and then transfected with U50, U50DTT or pSIREN-RetroQ control plasmid as described earlier. Forty-eight hours after transfection, cells were incubated with fresh medium containing ³H-thymidine for 4 h and were fixed and measured for ³H and ¹⁴C radioactivity. The ratio of ³H radioactivity to that of ¹⁴C indicates the rate of DNA synthesis or cell proliferation. Statistical significance was determined using Student's t-test. A P-value of 0.05 or smaller was considered statistically significant.

Expression constructs for LOC441164, NT5E and SYNCRIP were also transfected into LNCaP or 22Rv1 cells. Gene expression was confirmed by western blot analysis with anti-FLAG antibody, and the colony formation assay was conducted as described for U50 earlier. Two previously established growth-suppressor genes, FOXO1A and ATBF1, were used as the positive controls.

### Example 5

### Mutation Analysis

First amplified were the open-reading frames for the three protein-encoding genes, LOC441164, NT5E and SYNCRIP, from cDNA and snoRNAs U50 and U50′ sequence from genomic DNA by PCR from 15 prostate cancer cell lines and xenografts and directly sequenced the PCR products (Macrogen, Seoul, Republic of Korea). With the detection of the 2 bp deletion in U50, PCR was then performed in combination with SSCP in all the samples, as described previously (Sun et al., (2006) Prostate 66:660-666, incorporated herein by reference in its entirety). For a shifted band in a sample, which indicated a sequence alteration, another round of PCR-SSCP was performed to confirm the shift. Once a band shift was confirmed in a sample, genomic DNA of that sample was amplified and the PCR products were purified using the Qiaquick PCR Purification Kit (Qiagen, Germany) and sequenced to reveal the sequence alteration. For all samples including clinical samples and blood DNA samples, we also performed PCR combined with denaturing polyacrylamide gel electrophoresis to detect the TT deletion.

### Example 6

### Genotyping of the Prospective Cohort

DNA was extracted from buffy coat following standard protocols. For genotyping, each DNA sample was amplified by PCR using the same PCR primers for mutation detection in the presence of ³³P-dATP. PCR products were separated in a 35×45 cm² denaturing polyacrylamide sequencing gel, which was then dried and exposed to X-ray film to detect U50 alleles (the wild-type allele is 2-bases longer than the mutant allele). Blind duplicates (4%) were randomly interspersed with the case-control samples for quality control. Concordance for these quality control samples was 100%. The genotyping success rate was 100% for both case and control. The genotype distribution among controls was in Hardy-Weinberg equilibrium (P=0.64).

### Statistical Analysis in the Prospective Analysis of the Cohort

Both conditional and unconditional logistic regression models were used in the analysis of the association between the deletion and prostate cancer and observed consistent results with both approaches. To make use of information from all genotyped cases and controls, ORs were calculated using an unconditional logistic regression model that was adjusted for each of the matching variables rather than using a matched pair analysis. All models were adjusted for birth year (in single-year categories), blood collection date (in single-year categories) and race/ethnicity (white, African-American, Hispanic, Asian, other/unknown). Other covariates that were considered for the analysis were family history in a father and/or brother, education, smoking, diabetes, NSAID use, total calcium intake and PSA screening.

### Example 7

TABLE 1
Summary of U50 deletion in different tissue samples from prostate
cancer human subjects and men without cancer

| | Genotype^{a} distribution | | |
|---|---|---|---|
| | of U50 deletion | | |
| Samples (n) | −/− (%) | +/− (%) | +/+ (%) |
| Cancer xenografts and cell lines (30) | 2 (6.7) | 2 (6.7) | 26 (86.6) |
| Primary tumors from human subjects | 9 (10.1) | 5 (5.6) | 75 (84.3) |
| (89) | | | |
| Normal tissues from human subjects | 2 (2.2) | 9 (10.1) | 78 (87.7) |
| (89) | | | |
| Men without prostate cancer (104) | 0 (0) | 12 (11.5) | 92 (88.5) |

^{a}−/−, +/− and +/+ indicate homozygous, heterozygous and wild-type genotypes for the 2 bp deletion in U50 genome.

### Example 8

TABLE 2
ORs for total and clinically significant prostate cancer incidences determined by
homozygous (2/2) and heterozygous genotypes ( [symbol] /2) of the 2 bp deletion in
U50 at 6q14.3 in a prospective analysis

| | | | Clinically significant | |
|---|---|---|---|---|
| | All cases | | cases | |
| Genotype | Cases/Controls | OR^{a} (95% CI) | Cases/Controls | OR (95% CI) |
| +/+ | 1131/1131 | 1.00 (Reference) | 426/1131 | 1.00 (Reference) |
| +/− | 222/230 | 0.97 (0.79-1.19) | 98/230 | 1.15 (0.88-1.49) |
| −/− | 18/10 | 1.85 (0.85-4.03) | 10/10 | 2.63 (1.08-6.38) |

^{a}ORs on the basis of analysis adjusted for birth year, year of blood draw and race/ethnicity.
^{b}Clinically significant prostate cancer cases were defined by Gleason score ≧7 or grade 3-4, stage C or D at diagnosis or men who had prostate cancer as their underlying cause of death. When the 24 control men who were diagnosed with prostate cancer during follow-up were excluded in statistical analysis, the association between homozygous deletion and risk of total prostate cancer (OR 2.03; 95% CI 0.91-4.55) and clinically significant prostate cancer (OR 2.90; 95% CI 1.17-7.21) was stronger.

### Example 9

### SnoRNA U50 Inhibits Colony Formation in Breast Cancer Cells

To functionally evaluate the candidacy of U50 for the 6q tumor suppressor gene in breast cancer, a U50 expression plasmid was transfected, along with empty vector control, into the breast cancer cell lines MDA-MB-231 and Hs 578T, both of which express reduced levels of U50, and are homozygously mutated U50 (as shown in FIG. 20). A colony formation assay was then performed.

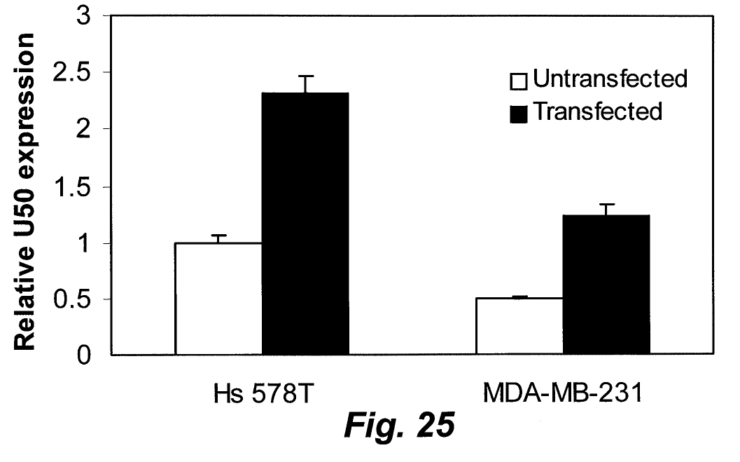

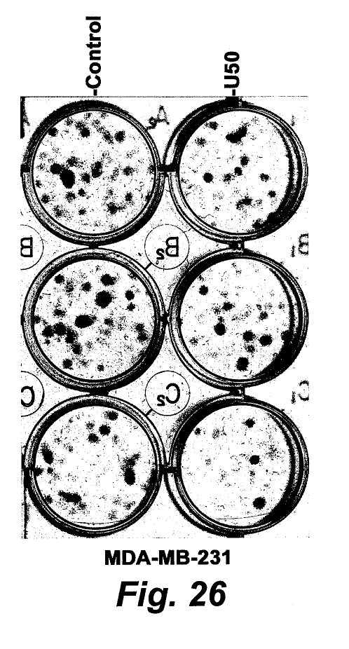

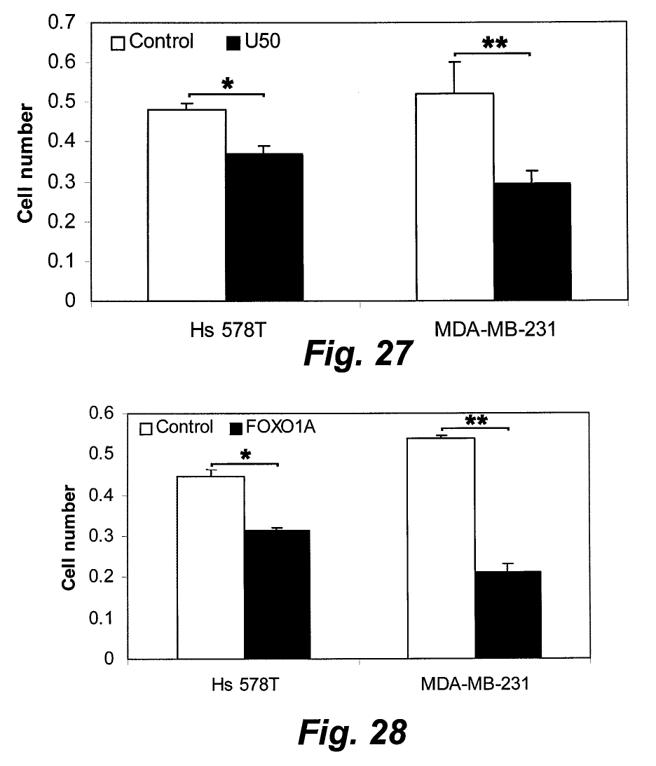

RNA expression of transfected U50 was confirmed by real time PCR analysis in transfected cells (FIG. 25). In both cell lines, ectopic wild-type U50 expression significantly reduced colony formation (FIGS. 26 and 27). As a positive control of colony formation assay, transfection of FLAG-pcDNA3-FOXO1A into both cell lines significantly inhibited colony formation, as shown in FIG. 28.

### Example 10

Association U50 Germline Mutation with Breast Cancer Risk

To evaluate whether germline deletion of U50 is associated with increased risk of breast cancer, as reported for such an association in prostate cancer (Dong et al. (2008) Hum. Mol. Genet. 17:1031-1042, U50 deletions in blood DNA samples from 395 human subjects with breast cancer, and 396 samples from control women, were genotyped. In these cases, 2 (0.5%) samples had germlne homozygous TT-deletion and 57 (14.4%) had hemizygous deletion, while the rest had wild-type U50. In the 396 control samples, 3 (0.8%) had a homozygous deletion of TT and 36 (9.1%) had a hemizygous deletion. While the numbers of samples with homozygous deletion were too small for comparison, human subjects had more frequent hemizygous deletions than did the control women (P=0.05, Chi-square test).

### Example 11

### Both Wildtype and Mutant Alleles of U50 are Expressed in Breast Cancer Cells

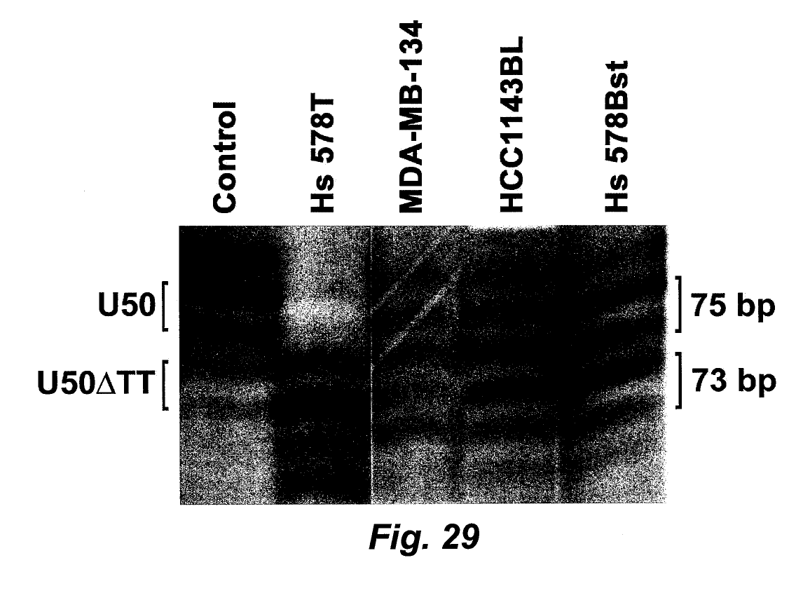

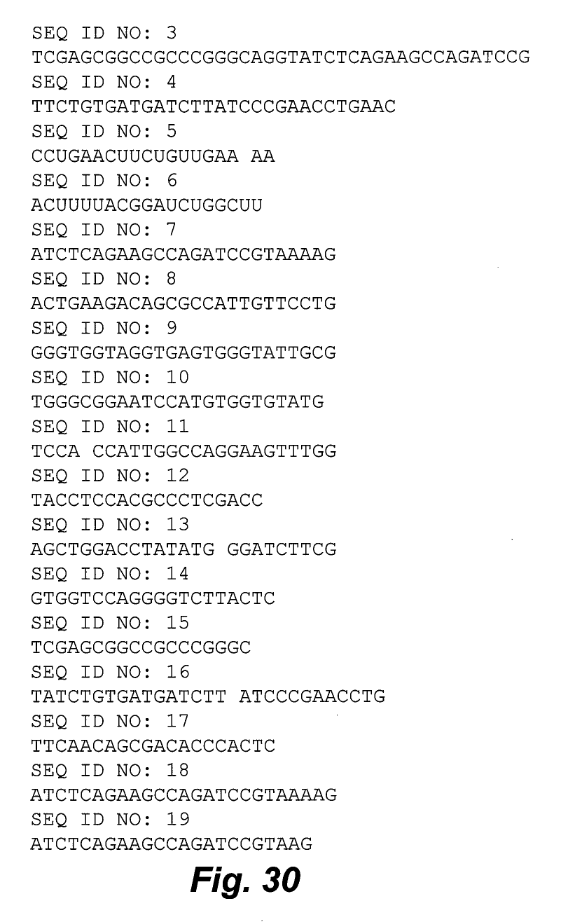

Certain samples analyzed, including cell lines MDA-MB-134, HCC1143BL and Hs 578Bst, showed a heterozygous genotype of the U50 deletion. To examine the question of whether both alleles or only one of the alleles, either wild-type or mutant, is expressed in these samples cDNA was transcribed from U50 RNA from these samples. U50 transcripts were amplified by PCR and sequenced the PCR products. Both wild-type and mutant U50 were expressed in these three samples (FIG. 29), indicating that neither allele has a preference in expression.

### Example 12

### Male-Female Differences

Compared to men, the frequency of this germline homozygous deletion in women seems to be lower (28/2236=1.25% versus 5/791=0.63%; P=0.16), and when compared to men, the frequency of the germline hemizygous deletion in women is significantly lower (Total: 447/2236=20% versus 93/791=11.8%; P=0.000).
(Human subject: 217/1106=19.6% versus 57/395=14.4%; P=0.02)
(Control: 230/1131=20.3% versus 36/396=9.1%; P=0.000)

TABLE 3
Allelic frequencies of U50 deletions in a cohort of 395 women with breast
cancer and 396 control women.

| Samples (n) | Wt (%)^{a} | Het (%)^{a} | Hom (%)^{a} |
|---|---|---|---|
| Control (396) | 357 (90.1) | 36 (9.1) | 3 (0.8) |
| Case (395) | 336 (85.1) | 57 (14.4)^{b} | 2 (0.5) |

^{a}Wt, het, and hom: wild type, heterozygous genotype, and homozygous genotype for the 2-bp deletion in the U50 gene.
^{b}P = 0.027, Fisher's exact test.

We claim:

1. A method of identifying a genetic marker of a human subject indicating a cancerous tissue in the human subject, the method comprising:
obtaining an isolated nucleic acid sample from a human subject; and
determining from the isolated nucleic acid sample the genotype of the human subject with respect to a gene locus encoding a snoRNA U50, whereby a mutation within the nucleotide sequence encoding a snoRNA U50, when compared with a wild-type nucleotide sequence encoding a snoRNA U50, identifies in the human subject a genetic marker associated with a cancer in the human subject.

2. The method according to claim 1, wherein the nucleotide sequence encoding a snoRNA U50 comprises the nucleotide sequence according to SEQ ID NO: 1.

3. The method according to claim 1, wherein the wild-type U50 nucleic acid sequence comprises the nucleotides 47-60 of the nucleotide sequence according to SEQ ID NO: 1.

4. The method according to claim 1, wherein the mutation is a TT dinucleotide deletion from within a nucleotide region comprising nucleotide position 47 to position 60 of the nucleotide sequence according to SEQ ID NO: 1, and wherein the mutation is associated with a cancer.

5. The method according to claim 1, wherein the cancer is a prostate cancer or a breast cancer.

6. The method according to claim 1, wherein the step of determining from the isolated nucleic acid the genotype of the biological sample with respect to a U50 locus encoding a snoRNA U50 comprises:
isolating by PCR amplification a nucleic acid molecule comprising the nucleotide sequence from nucleotide position 47 to position 60 of the nucleotide sequence according to SEQ ID NO: 1; and
determining whether the nucleic acid molecule has a dinucleotide deletion within the nucleotide sequence from nucleotide position 47 to position 60 of the nucleotide sequence according to SEQ ID NO: 1 when compared to a wild-type control nucleotide sequence.

7. The method according to claim 6, wherein the PCR amplification uses oligonucleotide primers having the nucleotide sequences according to SEQ ID NOs: 3 and 4.

8. The method according to claim 6, wherein determining whether the nucleic acid molecule has dinucleotide deletion within the nucleotide sequence from nucleotide position 47 to position 60 of the nucleotide sequence according to SEQ ID NO: 1 when compared to a wild-type control nucleotide sequence is by a single-base extension reaction.

9. The method according to claim 8, wherein the single-base extension reaction uses a primer having a nucleotide sequence selected from the group consisting of SEQ ID NOs: 3 and 4.

10. The method according to claim 1, wherein the isolated nucleic acid from the human subject and a first oligonucleotide probe having a nucleotide sequence capable of specifically detecting a mutation within a nucleotide sequence of the isolated nucleic acid encoding an snoRNA U50 are hybridized under conditions allowing the first probe to specifically hybridize to the isolated nucleic acid sample if the nucleotide sequence encoding the snoRNA U50 has a mutation therein with a cancer.

11. The method according to claim 10, wherein the first oligonucleotide probe comprises the nucleotide sequence according to SEQ ID NO: 19.

12. The method according to claim 1, wherein the first oligonucleotide is capable of specifically hybridizing under stringent conditions to a nucleic acid molecule comprising the nucleotide sequence according to SEQ ID NO: 2.

13. The method according to claim 10, further comprising hybridizing the isolated nucleic sample with a second oligonucleotide probe having a nucleotide sequence capable of specifically detecting under high stringency conditions a nucleotide sequence encoding an snoRNA U50, wherein the nucleotide sequence encoding the snoRNA U50 does not have a mutation therein with a cancer.

14. The method according to claim 13, wherein the second oligonucleotide comprises the nucleotide sequence according to SEQ ID NO: 18.

15. The method according to claim 1, further comprising correlating the presence of the genetic marker in the gene locus encoding the snoRNA U50 with the prognostic outcome for a prostate cancer in the human subject.

16. The method according to claim 1, further comprising correlating the presence of the genetic marker in the gene locus encoding the snoRNA U50 with the presence or absence of a breast cancer in the human subject.

17. The method according to claim 1, further comprising correlating the presence of a ΔTT genetic marker in the gene locus encoding the snoRNA U50 with a probability of the human subject developing a prostate or a breast cancer.

18. A method of modifying the proliferative status of a cell, comprising introducing into the cell a nucleic acid molecule comprising a sequence comprising the sequence of nucleotides from nucleotide position about 47 to about position 60 of the nucleotide sequence according to SEQ ID NO: 1.

19. The method according to claim 17, wherein the nucleic acid molecule comprises the nucleotide sequence according to SEQ ID NO: 1.

20. The method according to claim 17, wherein the introduction into the cell of the nucleic acid molecule reduces the proliferation of the cell.

21. The method according to claim 17, wherein the cell is selected from the group consisting of: a prostate cancer cell and a breast cancer cell.

22. A kit for determining whether a biological sample from a human subject has dinucleotide deletion within a nucleic acid region encoding the snoRNA U50, wherein the kit comprises at least one oligonucleotide comprising a nucleotide sequence selected from the group consisting of the nucleotide sequences according to SEQ ID NOs: 3, 4, 5, 6, 7, 18 and 19, and instructions for determining whether an isolated nucleic acid sample from a human subject has cancer-associated mutation within a nucleotide region encoding snoRNA U50.


SEQUENCE LISTING (19 sequences)

SEQ ID NO: 1 - RNA, 75 nt, Artificial sequence
  snoRNA U50
uaucugugaugaucuuaucccgaaccugaacuucuguugaaaaaaaaaaacuuuuacggaucuggcuucugagau

SEQ ID NO: 2 - RNA, 73 nt, Artificial sequence
  snoRNA U50DELTT
uaucugugaugaucuuaucccgaaccugaacuucuguugaaaaaaaaaaacuuacggaucuggcuucugagau

SEQ ID NO: 3 - DNA, 41 bp, Artificial sequence
  Linker-U50-specific forward primer
tcgagcggccgcccgggcaggtatctcagaagccagatccg

SEQ ID NO: 4 - DNA, 30 bp, Artificial sequence
  U50-specific reverse primer
ttctgtgatgatcttatcccgaacctgaac

SEQ ID NO: 5 - RNA, 19 nt, Artificial sequence
  siRNA No. 1
ccugaacuucuguugaaaa

SEQ ID NO: 6 - RNA, 19 nt, Artificial sequence
  siRNA No. 2
acuuuuacggaucuggcuu

SEQ ID NO: 7 - DNA, 25 bp, Artificial sequence
  U50-specific reverse primer
atctcagaagccagatccgtaaaag

SEQ ID NO: 8 - DNA, 25 bp, Artificial sequence
  LOC441164-specific forward primer
actgaagacagcgccattgttcctg

SEQ ID NO: 9 - DNA, 25 bp, Artificial sequence
  LOC441164-specific reverse primer
gggtggtaggtgagtgggtattgcg

SEQ ID NO: 10 - DNA, 24 bp, Artificial sequence
  NT5E-specific forward primer
tgggcggaatccatgtggtgtatg

SEQ ID NO: 11 - DNA, 24 bp, Artificial sequence
  NT5E-specific reverse primer
tccaccattggccaggaagtttgg

SEQ ID NO: 12 - DNA, 19 bp, Artificial sequence
  SYNCRIP-specific forward primer
tacctccacgccctcgacc

SEQ ID NO: 13 - DNA, 24 bp, Artificial sequence
  SYNCRIP-specific reverse primer
agctggacctatatgggatcttcg

SEQ ID NO: 14 - DNA, 20 bp, Artificial sequence
  GAPDH-specific forward primer
gtggtccaggggtcttactc

SEQ ID NO: 15 - DNA, 18 bp, Artificial sequence
  Linker-specific forward primer
tcgagcggccgcccgggc

SEQ ID NO: 16 - DNA, 28 bp, Artificial sequence
  U50-specific forward primer
tatctgtgatgatcttatcccgaacctg

SEQ ID NO: 17 - DNA, 20 bp, Artificial sequence
  GAPDH reverse primer
ttcaacagcgacacccactc

SEQ ID NO: 18 - DNA, 25 bp, Artificial sequence
  U50-specific probe
atctcagaagccagatccgtaaaag

SEQ ID NO: 19 - DNA, 23 bp, Artificial sequence
  U50DELTT-specific probe
atctcagaagccagatccgtaag